SEQUENCE LISTING (24 sequences)

SEQ ID NO: 1 - protein, 166 aa, synthetic construct
CDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNVDSILAVKKYFQRITLYLTEKKYSPCAWEVVRAEIMRSFSLSTNLQERLRRKE

SEQ ID NO: 2 - protein, 166 aa, synthetic construct
CDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNVDSILAVKKYFQRITLYLTEKKYSPCAWEVVRAEIMASFSLSTNLQERLRRKE

SEQ ID NO: 3 - protein, 166 aa, synthetic construct
CDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNVDSILAVKKYFQRITLYLTEKKYSPCAWEVVRDEIMRSFSLSTNLQERLRRKE

SEQ ID NO: 4 - protein, 166 aa, synthetic construct
CDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNVDSILAVKKYFQRITLYLTEKKYSPCAWEVVRKEIMRSFSLSTNLQERLRRKE

SEQ ID NO: 5 - protein, 166 aa, synthetic construct
CDLPQTHSLGNRRALILLAQMRRISPFSCLKDRHDFGFPQEEFDGNQFQKAQAISVLHEMIQQTFNLFSTKDSSAAWDESLLEKFYTELYQQLNDLEACVIQEVGVEETPLMNVDSILAVKKYFQRITLYLTEKKYSPCAWEVVRDEIMASFSLSTNLQERLRRKE

SEQ ID NO: 6 - protein, 165 aa, Homo sapiens [sequence omitted]

SEQ ID NO: 7 - protein, 165 aa, synthetic construct
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLRSKE

SEQ ID NO: 8 - protein, 166 aa, Homo sapiens
CDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMRSFSLSANLQERLRRKE

SEQ ID NO: 9 - protein, 166 aa, synthetic construct
CDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMASFSLSANLQERLRRKE

SEQ ID NO: 10 - protein, 166 aa, synthetic construct
CDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRDEIMRSFSLSANLQERLRRKE

SEQ ID NO: 11 - protein, 166 aa, Homo sapiens
CDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRAEIMRSFSSSRNLQERLRRKE

SEQ ID NO: 12 - protein, 166 aa, synthetic construct
CDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRAEIMASFSSSRNLQERLRRKE

SEQ ID NO: 13 - protein, 166 aa, synthetic construct
CDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRDEIMRSFSSSRNLQERLRRKE

SEQ ID NO: 14 - protein, 166 aa, synthetic construct
CDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRKEIMRSFSSSRNLQERLRRKE

SEQ ID NO: 15 - protein, 214 aa, synthetic construct
DIQMTQSPSSVSASVGDRVTITCKTSQDVNTAVAWYQQKPGQAPRLLIYWASTRHTGVPDRFSGSGSGTDFTLTISSLQAEDVAVYYCQQHYNTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 445 aa, synthetic construct
QMQLVQSGAEVKKTGSSVKVSCKASGYTFTSYSINWVRQAPGKGLEWVAYFYVGNGYTDYNEKFKGRVTMTRDTSTSTVYMELSSLRSEDTAVYYCARGGLPYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 444 aa, synthetic construct
QMQLVQSGAEVKKTGSSVKVSCKASGYTFTSYSINWVRQAPGKGLEWVAYFYVGNGYTDYNEKFKGRVTMTRDTSTSTVYMELSSLRSEDTAVYYCARGGLPYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - protein, 444 aa, synthetic construct
QMQLVQSGAEVKKTGSSVKVSCKASGYTFTSYSINWVRQAPGKGLEWVAYFYVGNGYTDYNEKFKGRVTMTRDTSTSTVYMELSSLRSEDTAVYYCARGGLPYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 19 - protein, 444 aa, synthetic construct
QMQLVQSGAEVKKTGSSVKVSCKASGYTFTSYSINWVRQAPGKGLEWVAYFYVGNGYTDYNEKFKGRVTMTRDTSTSTVYMELSSLRSEDTAVYYCARGGLPYYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 20 - protein, 6 aa, synthetic construct
SGGGGS

SEQ ID NO: 21 - protein, 14 aa, synthetic construct
AEAAAKEAAAKAGS

SEQ ID NO: 22 - protein, 8 aa, synthetic construct
GGGGGGGG

SEQ ID NO: 23 - protein, 15 aa, synthetic construct
GGGGSGGGGSGGGGS

SEQ ID NO: 24 - protein, 19 aa, synthetic construct
MGWSWILLFLLSVTAGVHS

(19) United States
(12) Patent Application Publication
(10) Pub. No.: US 2025/0145680 A1
(43) Pub. Date: May 8, 2025

(54) NOVEL INTERFERON VARIANTS AND BIFUNCTIONAL FUSION MOLECULES THEREOF

(71) Applicant: REMD Biotherapeutics, Inc., Camarillo, CA (US)

(72) Inventors: Hai Yan, Thousand Oaks, CA (US); Wuyi Wang, Newbury Park, CA (US); Kristopher Steward, Newbury Park, CA (US)

(73) Assignee: REMD Biotherapeutics, Inc., Camarillo, CA (US)

(21) Appl. No.: 18/684,361

(22) PCT Filed: Sep. 12, 2022
(86) PCT No.: PCT/US2022/040807

§ 371 Date: Feb. 16, 2024

Related U.S. Application Data
(60) Provisional application No. 63/347,871, filed on Jun. 1, 2022.
(60) Provisional application No. 63/234,498, filed on Aug. 18, 2021.

(51) Int. Cl. C07K 14/56; A61K 38/00; A61K 45/06; A61P 35/00; C07K 16/28

(52) U.S. Cl. CPC C07K 14/56; A61K 45/06; A61P 35/00; C07K 16/2827; A61K 38/00; C07K 2319/30; C07K 2319/33

(57) ABSTRACT

The present invention relates to bifunctional fusion molecules comprising a mutated polypeptide ligand (e.g., mutated IFN-α) having reduced biological activity attached to a disease tissue targeting biologic or a tumor associated antigen (TAA)-targeting biologic (e.g., anti-PD-L1 antibody), wherein the targeting biologic or TAA-targeting biologic directs the mutated ligands to cells that express on their surfaces the antigens to which said targeting biologic binds, as well as receptors for said ligands. Importantly, because the mutated polypeptide ligands have reduced biological activity, the resultant fusion molecule have reduced off-target activity/toxicity. More importantly, the targeting of the mutated ligand by the targeting biologic restores the activity of the mutated ligand, with the degree of activity restoration apparently correlated with the level of targeting biologic on the cells. As such, the fusion molecules of the present invention advance the state of art by providing bifunctional fusion molecules having a greater therapeutic window than those previously described. The invention further relates to methods of treating cancer in a patient and in particular, patients with refractory and/or recurrent cancers involving the use of these bifunctional fusion molecules. The invention also relates to methods of treating infectious disease, including but not limited to chronic hepatitis B and C infections in a patient involving the use of these bifunctional fusion molecules.

### RELATED PATENT APPLICATIONS

This application claims benefit of U.S. Provisional Application No. 63/234,498, filed on Aug. 18, 2021, and U.S. Provisional Application No. 63/347,871, filed on Jun. 1, 2022, each incorporated in its entirety by reference herein.

### TECHNICAL FIELD

Interferons (IFNs) are soluble proteins produced naturally by cells in response to viruses. Interferons include type 1 interferons (e.g., interferon-alpha (IFN-α) and interferon-beta (IFN-β)) and type 2 interferons (e.g., interferon-gamma (IFN-γ)). Although first described for their ability to inhibit viral replication, IFN-α's have multiple properties exhibiting anti-proliferative effects, induction of apoptosis (Rodriguez-Villanueva J and T J McDonnell, Int J Cancer, 61:110, 1995) and induction of the tumor suppressor gene, P53, in tumor cells (Takaoka A et al., Nature, 424:516, 2003). IFN-α's were the first recombinant proteins used for the treatment of various cancers and IFN-α has been approved by the FDA for the treatment of several cancers including melanoma, renal cell carcinoma, B cell lymphoma, multiple myeloma, chronic myelogenous leukemia (CML) and hairy cell leukemia. IFN-α, pegylated IFN-α, and consensus IFN (interferon alfacon-1) are also approved by the FDA for the treatment of chronic infection with hepatitis C (HCV) and/or hepatitis B (HBV) virus.

All type 1 IFNs are recognized by a shared receptor, IFN-αR, composed of two transmembrane proteins, IFN-αR1 and IFN-αR2. Type I interferon (IFN) signaling drives pathology in a number of autoimmune diseases, in particular in systemic lupus erythematosus (SLE), and can be tracked via type I IFN-inducible transcripts present in whole blood—said transcripts provide a type I IFN gene signature. By way of example, Yao et al. (Hum Genomics Proteomics 2009, pii: 374312) describe the identification of an IFNα/β 21-gene signature and its use as a biomarker of type I IFN-related diseases or disorders. A “direct” effect of IFN-α on the tumor cells is mediated by the IFN-α binding directly to the type I IFN receptor on those cells and stimulating apoptosis, terminal differentiation or reduced proliferation. Unfortunately, the type I interferon receptor is also present on most non-cancerous and non-virally infected cells. Systemic activation of this receptor on such cells by IFN-α causes the expression of numerous pro-inflammatory cytokines and chemokines, leading to toxicity. Such toxicity prevents the dosing of IFN-α to a subject at levels that exert the maximum anti-proliferative and pro-apoptotic activity on the cancer cells, and the use of IFN-α to treat cancer has been limited by its short half-life and associated systemic toxicities (Weiss K, Semin Oncol, 25:9, 1998; Jones G J and Itri L M, Cancer, 57:1709, 2006). The limitations of systemic IFN-α therapy have led to the exploration of alternative strategies to deliver IFN-α safely and effectively into the site of tumor or viral infection.

Cancer immunotherapy is the name given to cancer treatments that use the immune system to attack cancers and is rapidly evolving from therapies that globally and non-specifically simulate the immune system to more targeted activation of individual components of the immune system, resulting in increased efficacy and decreased toxicity. Of primary focus are therapies that inhibit the interaction between Programmed Death Ligand 1 (PD-L1), present on the surface of tumor or antigen-presenting cells, and Programmed Death 1 (PD-1), present on the surface of activated lymphocytes. Programmed Death 1 (PD-1) is a member of the CD28 family of receptors, which also includes CD28, CTLA-4, ICOS, and BTLA. Through its binding to its two main ligands PD-L1 (B7-H1) or PD-L2 (B7-DC), PD-1 down-regulates T-cell activation that would typically occur with recognition, by the T cell regulators, of tumor antigens expressed in the context of the MHC on tumor cells. In cancer, the PD-L1/PD-1 pathways can protect tumors from cytotoxic T cells, ultimately inhibiting the antitumor immune response by deactivating cytotoxic T cells in the tumor microenvironment and preventing priming and activation of new T cells in the lymph nodes and subsequent recruitment to the tumor (Chen and Irving, Clin Cancer Res., 18:6580-6587, 2012).

Inhibition of the PD-1/PD-L1 interaction mediates potent antitumor activity in preclinical models (U.S. Pat. Nos. 8,008,449 and 7,943,743), and the use of Ab inhibitors of the PD-1/PD-L1 interaction for treating cancer has become standard of care for many types of cancer (see, e.g., Topalian et al., Curr Opin Immunol., 24:207-212, 2012; Brahmer et al., N Engl J Med., 366 (26):2455-65, 2012; Garon et al., N Engl J Med, 372:2018-2028, 2015; Philips et al., Int. Immunol., 27(1):39-46, 2015; Migden et al., N Engl J Med, 379:341-351, 2018). PD-1 expression has been found on tumor infiltrating T cells and PD-L1 expression has been found on tumor cells and myeloid cells within the tumor in many murine and human cancers, including human lung, ovarian and colon carcinoma and various myelomas, and anti-PD-1 and anti-PD-L1 antibodies developed by, e.g., Bristol-Myers Squibb (nivolumab), Merck (pembrolizumab), Regeneron (cemiplimab), Roche (atezolizumab), AstraZeneca (durvalumab) have been approved by the FDA to treat various cancer indications. The tolerability of PD-1-pathway blockers and their unique mechanism of action have made them ideal backbones for combination regimen development. Recent clinical data combining CTLA-4 and PD-1 blockade in melanoma patients showed an increased rate of objective tumor responses as compared to blocking either checkpoint alone, supporting the notion that combinatorial checkpoint blockade may result in increased clinical benefit (Wolchok et al., N Engl J Med, 366:2443-54, 2012). The combination of Yervoy and Opdivo has been approved for treatment of certain patients with melanoma, mesothelioma, non-small cell lung cancer, hepatocellular carcinoma, colorectal cancer, and rental cell carcinoma.

The PD-1/PD-L1 immune checkpoint is also involved in chronic viral infections, including hepatitis B virus (HBV) and hepatitis C virus (HCV). The chronic inflammation in the liver due to viral infection results in the expression of PD-L1 on hepatocytes, which supports the persistence of infection due to down-regulation of the anti-viral immune response via negative signaling through PD-1 expressed by immune cells. PD-1 and PD-L1 blocking antibodies are being tested in the clinic for chronic HBV and HCV infection and have shown promising activity. The proposed mechanism of action for these antibodies is restoring the anti-viral activity of dysfunctional immune cells at the site of infection.

The present invention relates to bifunctional fusion molecules comprising a mutated polypeptide ligand (e.g., mutated IFN-α) having reduced biological activity attached to a disease tissue targeting biologic or a tumor associated antigen (TAA)-targeting biologic (e.g., anti-PD-L1 antibody), wherein the targeting biologic or TAA-targeting biologic directs the mutated ligands to cells that express on their surfaces the antigens to which said targeting biologic binds, as well as receptors for said ligands. Importantly, because the mutated polypeptide ligands have reduced biological activity, the resultant fusion molecule have reduced off-target activity and off-target toxicity. More importantly, the targeting of the mutated ligand by the targeting biologic restores the activity of the mutated ligand, with the degree of activity restoration apparently correlated with the level of targeting biologic on the cells.

As such, the fusion molecules of the present invention advance the state of art by providing bifunctional fusion molecules having a greater concentration therapeutic window than those previously described.

### DISCLOSURE OF THE INVENTION

In one aspect, the present invention relates to the production of mutated variants of consensus IFN-α (con-IFN-α). These variants have amino acid substitutions which reduce their affinity for the IFN-αR1 and IFN-αR2 receptor complex (IFN-αR) and reduced or abolished ability to activate IFN-αR expressing cells but retain the ability to bind IFN-αR and the ability to bind and activate the IFN-αR receptor complex. In various embodiments, the mutant con-IFN-α has a biological activity selected from less than 70% of the biological activity of the wild-type con-IFN-α; less than 60% of the biological activity of the wild-type con-IFN-α; less than 50% of the biological activity of the wild-type con-IFN-α; less than 40% of the biological activity of the wild-type con-IFN-α; less than 30% of the biological activity of the wild-type con-IFN-α; less than 20% of the biological activity of the wild-type con-IFN-α; or less than 10% of the biological activity of the wild-type of which it is deduced (i.e., the wild-type con-IFN-α of which the coding sequence has been mutated to obtain the mutant IFN).

In another aspect, the present invention relates to the use of these mutated con-IFN-α variants to construct bifunctional fusion molecules comprising a mutated con-IFN-α variant attached to a disease tissue targeting moiety or a tumor associated antigen (TAA)-targeting moiety, wherein the targeting moiety directs the mutated con-IFN-α variant to cells that express on their surfaces the antigens to which said targeting moiety binds, as well as receptors for said ligands. In various embodiments, the targeting moiety is targeting to a marker expressed on an IFN receptor-expressing cell. In various embodiments, the targeting moiety is directed to a tissue-specific marker. In various embodiments, the tissue is a cancer tissue. In various embodiments, the bifunctional fusion molecules comprise a targeting moiety in the form of an antibody, a bispecific antibody, a heterodimeric antibody, an antibody fragment, a diabody, a protein or a peptide binding to a molecule enriched in the cancer tissue, such as a tumor associated antigen antibody (TAA Ab).

In another aspect, the present invention relates to the use of these mutated con-IFN-α variants to construct bifunctional fusion molecules comprising a mutated con-IFN-α variant attached to a disease tissue targeting moiety or a tumor associated antigen (TAA)-targeting moiety, wherein the targeting moiety directs the mutated con-IFN-α variant to cells that express on their surfaces the antigens to which said targeting moiety binds, as well as receptors for said ligands. In various embodiments, the targeting moiety is targeting to a marker expressed on an IFN receptor-expressing cell. In various embodiments, the targeting moiety is directed to a virally infected tissue-specific marker. In various embodiments, the tissue is a hepatitis virus infected liver. In various embodiments, the bifunctional fusion molecules comprise a targeting moiety in the form of an antibody, a bispecific antibody, a heterodimeric antibody, an antibody fragment, a diabody, a protein or a peptide binding to a molecule enriched in the virus infected liver tissue.

In various embodiments, the fusion molecule comprises a type 1 interferon molecule. In various embodiments, the fusion molecule comprises an interferon-alpha (IFN-α) molecule. In various embodiments, the fusion molecule comprises an interferon-beta (IFN-β) molecule. In various embodiments, the fusion molecule comprises a consensus interferon-alpha (con-IFN-α) molecule. In various embodiments, the fusion molecule comprises a type 1 interferon mutant molecule. In various embodiments, the fusion molecule comprises a con-IFN-α molecule having the amino acid sequence of SEQ ID NO: 1. In various embodiments, the fusion molecule comprises a con-IFN-α mutant molecule having the amino acid sequence of SEQ ID NO: 2. In various embodiments, the fusion molecule comprises a con-IFN-α mutant molecule having the amino acid sequence of SEQ ID NO: 3. In various embodiments, the fusion molecule comprises a con-IFN-α mutant molecule having the amino acid sequence of SEQ ID NO: 4. In various embodiments, the fusion molecule comprises a con-IFN-α mutant molecule having the amino acid sequence of SEQ ID NO: 5. In various embodiments, the fusion molecule comprises a human IFN-α2b molecule having the amino acid sequence of SEQ ID NO: 6. In various embodiments, the fusion molecule comprises a human IFN-α2b mutant molecule having the amino acid sequence of SEQ ID NO: 7. In various embodiments, the fusion molecule comprises a human IFN-α5 molecule having the amino acid sequence of SEQ ID NO: 8. In various embodiments, the fusion molecule comprises a human IFN-α5 mutant molecule having the amino acid sequence of SEQ ID NO: 9. In various embodiments, the fusion molecule comprises a human IFN-α5 mutant molecule having the amino acid sequence of SEQ ID NO: 10. In various embodiments, the fusion molecule comprises a human IFN-α6 molecule having the amino acid sequence of SEQ ID NO: 11. In various embodiments, the fusion molecule comprises a human IFN-α6 mutant molecule having the amino acid sequence of SEQ ID NO: 12. In various embodiments, the fusion molecule comprises a human IFN-α6 mutant molecule having the amino acid sequence of SEQ ID NO: 13. In various embodiments, the fusion molecule comprises a human IFN-α6 mutant molecule having the amino acid sequence of SEQ ID NO: 14.

In various embodiments, the fusion molecule comprises an TAA Ab selected from the group consisting of a fully human antibody, a humanized antibody, a chimeric antibody, a monoclonal antibody, a polyclonal antibody, a recombinant antibody, a bispecific antibody, a heterodimeric antibody, an antigen-binding antibody fragment, a Fab, a Fab′, a Fab₂, a Fab′₂, a IgG, a IgM, a IgA, a IgE, a scFv, a dsFv, a dAb, a nanobody, a unibody, and an diabody. In various embodiments, the antibody is a chimeric antibody. In various embodiments, the antibody is a humanized monoclonal antibody. In various embodiments, the antibody is a fully human monoclonal antibody. In various embodiments, the TAA Ab is a human anti-PD-L1 Ab. In various embodiments, the TAA Ab is a human anti-PD-1 Ab. In various embodiments, the TAA Ab is an anti-PD-L1 having the light chain sequence set forth in SEQ ID NO: 15 and the heavy chain sequence set forth in SEQ ID NO: 16. In various embodiments, the TAA Ab is an anti-PD-L1 having the light chain sequence set forth in SEQ ID NO: 15 and the heavy chain sequence set forth in SEQ ID NO: 17. In various embodiments, the TAA Ab is an anti-PD-L1 heterodimer having the light chain sequence set forth in SEQ ID NO: 15 and the heavy chain sequences set forth in SEQ ID NO: 18 and 19.

In various embodiments, the fusion molecule is a recombinantly expressed fusion molecule. In various embodiments, the fusion molecules comprise an interferon molecule that is directly attached to the targeting moiety. In various embodiments, the fusion molecules comprise an IFN molecule that is attached to the targeting moiety via a peptide linker. In various embodiments, the linker may be an artificial sequence of between 5, 10, 15, 20, 30, 40 or more amino acids that are relatively free of secondary structure. In various embodiments, the linker is a rigid linker peptide of between 10, 15, 20, 30, 40 or more amino acids that display α-helical conformation and may act as rigid spacers between protein domains. In various embodiments, the peptide linker is a G/S rich linker. In various embodiments, the peptide linker is an alpha-helical linker. In various embodiments, the peptide linker is a glycine linker. In various embodiments, the peptide linker has the sequence set forth in SEQ ID NO: 20. In various embodiments, the peptide linker has the sequence set forth in SEQ ID NO: 21. In various embodiments, the peptide linker has the sequence set forth in SEQ ID NO: 22. In various embodiments, the peptide linker has the sequence set forth in SEQ ID NO: 23.

In various embodiments, the fusion molecule is an anti-PD-L1 Ab-IFN-α2b fusion molecule (“FP-01”) comprising a light chain having the amino acid sequence set forth in SEQ ID NO: 15, and a IFN-α2b molecule having the amino acid sequence of SEQ ID NO: 6 directly fused to the C-terminus of a heavy chain having the amino acid sequence set forth in SEQ ID NO: 16. In various embodiments, the fusion molecule is an anti-PD-L1 Ab-IFN-α2b (R149A) fusion molecule (“FP-02”) comprising a light chain having the amino acid sequence set forth in SEQ ID NO: 15, and a IFN-α2b (R149A) molecule having the amino acid sequence of SEQ ID NO: 7 directly fused to the C-terminus of a heavy chain having the amino acid sequence set forth in SEQ ID NO: 16. In various embodiments, the fusion molecule is an anti-PD-L1 Ab-IFN-α5 fusion molecule (“FP-03”) comprising a light chain having the amino acid sequence set forth in SEQ ID NO: 15, and a IFN-α5 molecule having the amino acid sequence of SEQ ID NO: 8 directly fused to the C-terminus of a heavy chain having the amino acid sequence set forth in SEQ ID NO: 16. In various embodiments, the fusion molecule is an anti-PD-L1 Ab-IFN-α5 (R150A) fusion molecule (“FP-04”) comprising a light chain having the amino acid sequence set forth in SEQ ID NO: 15, and a IFN-α5 (R150A) molecule having the amino acid sequence of SEQ ID NO: 9 directly fused to the C-terminus of a heavy chain having the amino acid sequence set forth in SEQ ID NO: 16. In various embodiments, the fusion molecule is an anti-PD-L1 Ab-IFN-α5 (A146D) fusion molecule (“FP-05”) comprising a light chain having the amino acid sequence set forth in SEQ ID NO: 15, and a IFN-α5 (A146D) molecule having the amino acid sequence of SEQ ID NO: 10 directly fused to the C-terminus of a heavy chain having the amino acid sequence set forth in SEQ ID NO: 16. In various embodiments, the fusion molecule is an anti-PD-L1 Ab-con-IFN-α fusion molecule (“FP-06”) comprising a light chain having the amino acid sequence set forth in SEQ ID NO: 15, and a con-IFN-α molecule having the amino acid sequence of SEQ ID NO: 1 directly fused to the C-terminus of a heavy chain having the amino acid sequence set forth in SEQ ID NO: 16. In various embodiments, the fusion molecule is an anti-PD-L1 Ab-con-IFN-α (R150A) fusion molecule (“FP-07”) comprising a light chain having the amino acid sequence set forth in SEQ ID NO: 15, and a con-IFN-α (R150A) molecule having the amino acid sequence of SEQ ID NO: 2 directly fused to the C-terminus of a heavy chain having the amino acid sequence set forth in SEQ ID NO: 16. In various embodiments, the fusion molecule is an anti-PD-L1 Ab-con-IFN-α (A146D) fusion molecule (“FP-08”) comprising a light chain having the amino acid sequence set forth in SEQ ID NO: 15, and a con-IFN-α (A146D) molecule having the amino acid sequence of SEQ ID NO: 3 directly fused to the C-terminus of a heavy chain having the amino acid sequence set forth in SEQ ID NO: 16. In various embodiments, the fusion molecule is an anti-PD-L1 Ab-con-IFN-α (A146K) fusion molecule (“FP-09”) comprising a light chain having the amino acid sequence set forth in SEQ ID NO: 15, and a con-IFN-α (A146K) molecule having the amino acid sequence of SEQ ID NO: 4 directly fused to the C-terminus of a heavy chain having the amino acid sequence set forth in SEQ ID NO: 16. In various embodiments, the fusion molecule is an anti-PD-L1 Ab-IFN-α6 fusion molecule (“FP-10”) comprising a light chain having the amino acid sequence set forth in SEQ ID NO: 15, and a IFN-α6 molecule having the amino acid sequence of SEQ ID NO: 11 directly fused to the C-terminus of a heavy chain having the amino acid sequence set forth in SEQ ID NO: 16. In various embodiments, the fusion molecule is an anti-PD-L1 Ab-IFN-α6 (R150A) fusion molecule (“FP-11”) comprising a light chain having the amino acid sequence set forth in SEQ ID NO: 15, and a IFN-α6 (R150A) molecule having the amino acid sequence of SEQ ID NO: 12 directly fused to the C-terminus of a heavy chain having the amino acid sequence set forth in SEQ ID NO: 16. In various embodiments, the fusion molecule is an anti-PD-L1 Ab-IFN-α6 (A146D) fusion molecule (“FP-12”) comprising a light chain having the amino acid sequence set forth in SEQ ID NO: 15, and a IFN-α6 (A146D) molecule having the amino acid sequence of SEQ ID NO: 13 directly fused to the C-terminus of a heavy chain having the amino acid sequence set forth in SEQ ID NO: 16. In various embodiments, the fusion molecule is an anti-PD-L1 Ab-IFN-α6 (A146K) fusion molecule (“FP-13”) comprising a light chain having the amino acid sequence set forth in SEQ ID NO: 15, and a IFN-α6 (A146K) molecule having the amino acid sequence of SEQ ID NO: 14 directly fused to the C-terminus of a heavy chain having the amino acid sequence set forth in SEQ ID NO: 16. In various embodiments, the fusion molecule is an anti-PD-L1 Ab-con-IFN-α fusion molecule (“FP-14”) comprising a light chain having the amino acid sequence set forth in SEQ ID NO: 15, and a con-IFN-α molecule having the amino acid sequence of SEQ ID NO: 2 fused to the C-terminus of a heavy chain having the amino acid sequence set forth in SEQ ID NO: 17 with a peptide linker having the sequence set forth in SEQ ID NO: 22. In various embodiments, the fusion molecule is an anti-PD-L1 Ab-con-IFN-α fusion molecule (“FP-15”) comprising a light chain having the amino acid sequence set forth in SEQ ID NO: 15, and a con-IFN-α molecule having the amino acid sequence of SEQ ID NO: 2 fused to the C-terminus of a heavy chain having the amino acid sequence set forth in SEQ ID NO: 17 with a peptide linker having the sequence set forth in SEQ ID NO: 23. In various embodiments, the fusion molecule is an anti-PD-L1 Ab-con-IFN-α fusion molecule (“FP-16”) comprising a light chain having the amino acid sequence set forth in SEQ ID NO: 15, and a con-IFN-α molecule having the amino acid sequence of SEQ ID NO: 3 fused to the C-terminus of a heavy chain having the amino acid sequence set forth in SEQ ID NO: 17 with a peptide linker having the sequence set forth in SEQ ID NO: 22. In various embodiments, the fusion molecule is an anti-PD-L1 Ab-con-IFN-α fusion molecule (“FP-17”) comprising a light chain having the amino acid sequence set forth in SEQ ID NO: 15, and a con-IFN-α molecule having the amino acid sequence of SEQ ID NO: 3 fused to the C-terminus of a heavy chain having the amino acid sequence set forth in SEQ ID NO: 17 with a peptide linker having the sequence set forth in SEQ ID NO: 23. In various embodiments, the fusion molecule is an anti-PD-L1 Ab-con-IFN-α fusion molecule (“FP-18”) comprising a light chain having the amino acid sequence set forth in SEQ ID NO: 15, and a con-IFN-α molecule having the amino acid sequence of SEQ ID NO: 5 fused to the C-terminus of a heavy chain having the amino acid sequence set forth in SEQ ID NO: 17 with a peptide linker having the sequence set forth in SEQ ID NO: 22. In various embodiments, the fusion molecule is an anti-PD-L1 Ab-con-IFN-α fusion molecule (“FP-19”) comprising a light chain having the amino acid sequence set forth in SEQ ID NO: 15, and a con-IFN-α molecule having the amino acid sequence of SEQ ID NO: 2 fused to the C-terminus of a heavy chain having the amino acid sequence set forth in SEQ ID NO: 19 with a peptide linker having the sequence set forth in SEQ ID NO: 22 and a second heavy chain having the amino acid sequence set forth in SEQ ID NO: 18 forming a heterodimeric antibody molecule. In various embodiments, the fusion molecule is an anti-PD-L1 Ab-con-IFN-α fusion molecule (“FP-20”) comprising a light chain having the amino acid sequence set forth in SEQ ID NO: 15, and a con-IFN-α molecule having the amino acid sequence of SEQ ID NO: 5 fused to the C-terminus of a heavy chain having the amino acid sequence set forth in SEQ ID NO: 19 with a peptide linker having the sequence set forth in SEQ ID NO: 22 and a second heavy chain having the amino acid sequence set forth in SEQ ID NO: 18 forming a heterodimeric antibody molecule.

In another aspect, the present invention provides a pharmaceutical composition which comprises a bifunctional fusion molecule of the present invention as an active ingredient, in a pharmaceutically acceptable excipient or carrier. In various embodiments, the pharmaceutical composition is formulated for administration via a route selected from the group consisting of subcutaneous injection, intraperitoneal injection, intramuscular injection, intrasternal injection, intravenous injection, intraarterial injection, intrathecal injection, intraventricular injection, intraurethral injection, intracranial injection, intrasynovial injection or via infusions.

In another aspect, the present disclosure relates to the identification and use of biomarkers for the detection and/or monitoring of a subject suffering from a type I interferon-mediated disease or disorder. In various embodiments, the disease or disorder is selected from the group consisting of: cancer, infectious diseases, immune disorders, inflammatory diseases or conditions, and autoimmune diseases.

In another aspect, the present disclosure provides a method for treating cancer or cancer metastasis in a subject, comprising administering a therapeutically effective amount of the pharmaceutical compositions of the invention to a subject in need thereof. In one embodiment, the subject is a human subject.

In another aspect, the present disclosure provides a method for treating cancer or cancer metastasis in a subject, comprising administering a therapeutically effective amount of the pharmaceutical compositions of the invention in combination with a second therapy selected from the group consisting of: cytotoxic chemotherapy, immunotherapy, small molecule kinase inhibitor targeted therapy, surgery, radiation therapy, and stem cell transplantation. In various embodiments, the combination therapy may comprise administering to the subject a therapeutically effective amount of immunotherapy, including, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, PD-L1, OX-40, CD137, GITR, LAG3, TIM-3, CD40, CD47, SIRPα, ICOS, Siglec 8, Siglec 9, Siglec 15, TIGIT and VISTA; treatment using bispecific T cell engaging antibodies (BiTE®) such as blinatumomab: treatment involving administration of biological response modifiers such as TNF family, IL-1, IL-4, IL-7, IL-12, IL-15, IL-17, IL-21, IL-22, GM-CSF, IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR: TLR7, TLR8, and TLR 9) agonists CpG and imiquimod; wherein the combination therapy provides increased effector cell killing of tumor cells, i.e., a synergy exists between the IFN variants and the immunotherapy when co-administered.

In various embodiments, the cancer is selected from the group consisting of: B cell lymphoma; a lung cancer (small cell lung cancer and non-small cell lung cancer); a bronchus cancer; a colorectal cancer; a prostate cancer; a breast cancer; a pancreas cancer; a stomach cancer; an ovarian cancer; a urinary bladder cancer; a brain or central nervous system cancer; a peripheral nervous system cancer; an esophageal cancer; a cervical cancer; a melanoma; a uterine or endometrial cancer; a cancer of the oral cavity or pharynx; a liver cancer; a kidney cancer; a biliary tract cancer; a small bowel or appendix cancer; a salivary gland cancer; a thyroid gland cancer; a adrenal gland cancer; an osteosarcoma; a chondrosarcoma; a liposarcoma; a testes cancer; and a malignant fibrous histiocytoma; a skin cancer; a head and neck cancer; lymphomas; sarcomas; multiple myeloma; and leukemias.

In various embodiments, the individual previously responded to treatment with an anti-cancer therapy, but, upon cessation of therapy, suffered relapse (hereinafter “a recurrent cancer”). In various embodiments, the individual has a resistant or refractory cancer.

In another aspect, the present disclosure provides a method for treating an infectious disease in a subject, comprising administering a therapeutically effective amount of the pharmaceutical compositions of the invention to a subject in need thereof. In one embodiment, the subject is a human subject.

In another aspect, the present disclosure provides a method for treating HBV infection in a subject, comprising administering a therapeutically effective amount of the pharmaceutical compositions of the invention to a subject in need thereof. In one embodiment, the subject is a human subject.

In another aspect, the present disclosure provides a method for treating HBV infection in a subject, comprising administering a therapeutically effective amount of the pharmaceutical compositions of the invention in combination with a second therapy selected from the group consisting of: treatment using nucleo(t)side analogs such as Tenofovir disoproxil fumarate (TDF), Tenofovir alafenamide (TAF), Lamivudine, Adefovir dipivoxil, Entecavir (ETV), Telbivudine, AGX-1009, emtricitabine, clevudine, ritonavir, dipivoxil, lobucavir, famvir, FTC, N-Acetyl-Cysteine (NAC), PC1323, theradigm-HBV, thymosin-alpha, and ganciclovir, besifovir (ANA-380/LB-80380), and tenofovir-exaliades (TLX/CMX157); treatment using other antiviral drugs such as siRNA, antisense oligonucleotides, capsid assembly modulators, and polymerase inhibitors; treatment using immunomodulatory therapies such as TLR7 agonist, TLR8 agonist, STING agonist, RIG-I activator, PD-1 blocking antibody, PD-L1 blocking antibody, TIM-3 blocking antibody, LAG-3 blocking antibody, and CTLA-4 blocking antibody; treatment using therapeutic vaccines against HBV antigens; and treatment using adoptive cellular therapy such as HBV-specific CAR-T cells, HBV-specific TCR-T cells, and other HBV-specific cellular therapies.

In another aspect, the present disclosure provides a method for treating HCV infection in a subject, comprising administering a therapeutically effective amount of the pharmaceutical compositions of the invention to a subject in need thereof. In one embodiment, the subject is a human subject.

In another aspect, the present disclosure provides a method for treating HCV infection in a subject, comprising administering a therapeutically effective amount of the pharmaceutical compositions of the invention in combination with a second therapy selected from the group consisting of protease inhibitors, polymerase inhibitors, direct-acting antivirals, ribavirin and pegylated interferon. In various embodiments, the second therapy is selected from the group consisting of: Mavyet (glecaprevir/pibrentasvir), Epclusa (sofosbuvir/velpatasvir), Vosevi (sofosbuvir/velpatasvir/voxilapresvir), Harvoni (ledipasvir/sofosbuvir), Sovaldi (sofosbuvir), and Zepatier (elbasvir/grazoprevir).

In other aspects, the present disclosure provides polynucleotides that encode the fusion molecules of the present disclosure; vectors comprising polynucleotides encoding fusion molecules of the disclosure; optionally, operably-linked to control sequences recognized by a host cell transformed with the vector; host cells comprising vectors comprising polynucleotides encoding fusion molecules of the disclosure; a process for producing a fusion molecule of the disclosure comprising culturing host cells comprising vectors comprising polynucleotides encoding fusion molecules of the disclosure such that the polynucleotide is expressed; and, optionally, recovering the fusion molecule from the host cell culture medium.

### BRIEF DESCRIPTION OF THE DRAWINGS

FIG. 1 is a set of diagrams showing the molecular structure of the antibody-IFN fusion proteins tested in these studies. A. comprises an antibody directly fused to an IFN molecule at the C-terminus of the antibody heavy chain. B. comprises an antibody fused to an IFN molecule at the C-terminus of the antibody heavy chain with a peptide linker. C. comprises a heterodimeric antibody using knob-into-hole and engineered disulfide bond technology where only one heavy chain is fused to an IFN molecule at the C-terminus with a peptide linker.

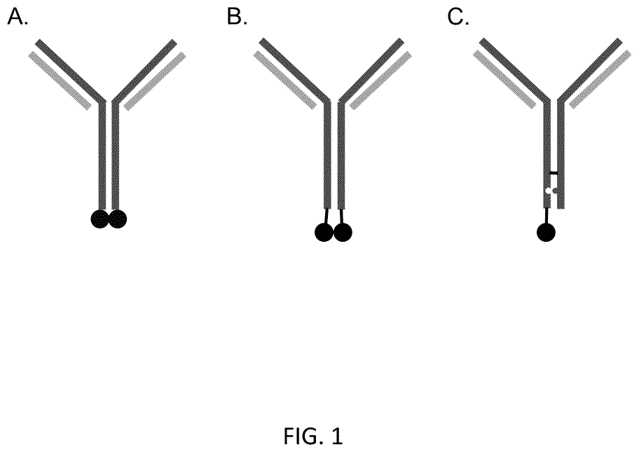

FIG. 2 is a line graph depicting the results of a cell growth inhibition assay. The cell growth inhibition was measured using CellTiter 96 AQueous Non-Radioactive Cell Proliferation Assay (Promega).

FIG. 3 is a line graph depicting the results of an MHC-1 expression assay. OVCAR-3 cells were incubated for 2 days in growth medium (cell control) or growth medium with the addition of samples (REMD290, IFN-α2b or fusion proteins). MHC-I expression was determined by FACS with PE anti-human HLA-A,B,C antibody (sample MFI) or without antibody (background MFI).

FIG. 4 is a line graph depicting the results of an PD-L1 expression assay. OVCAR-3 cells were incubated for 2 days in growth medium (cell control) or growth medium with addition of samples (REMD290, IFN-α2b or fusion proteins). PD-L1 expression was determined by FACS with REMD290 (sample MFI) or without antibody (background MFI), and then by FITC goat anti-human IgG secondary antibody.

### MODE(S) FOR CARRYING OUT THE INVENTION

### Definitions

The terms “polypeptide”, “peptide” and “protein” are used interchangeably herein to refer to a polymer of amino acid residues. In various embodiments, “peptides”, “polypeptides”, and “proteins” are chains of amino acids whose alpha carbons are linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free α-amino group on an amino acid at the amino terminal of a peptide or to the α-amino group (imino group when participating in a peptide bond) of an amino acid at any other location within the peptide. Similarly, the term “carboxy terminus” refers to the free carboxyl group on the carboxy terminus of a peptide or the carboxyl group of an amino acid at any other location within the peptide. Peptides also include essentially any polyamino acid including, but not limited to, peptide mimetics such as amino acids joined by an ether as opposed to an amide bond.

Polypeptides of the disclosure include polypeptides that have been modified in any way and for any reason, for example, to: (1) reduce susceptibility to proteolysis, (2) reduce susceptibility to oxidation, (3) alter binding affinity for forming protein complexes, (4) alter binding affinities, and (5) confer or modify other physicochemical or functional properties.

An amino acid “substitution” as used herein refers to the replacement in a polypeptide of one amino acid at a particular position in a parent polypeptide sequence with a different amino acid. Amino acid substitutions can be generated using genetic or chemical methods well known in the art. For example, single or multiple amino acid substitutions (e.g., conservative amino acid substitutions) may be made in the naturally occurring sequence (e.g., in the portion of the polypeptide outside the domain(s) forming intermolecular contacts). A “conservative amino acid substitution” refers to the substitution in a polypeptide of an amino acid with a functionally similar amino acid. The following six groups each contain amino acids that are conservative substitutions for one another:
1) Alanine (A), Serine(S), and Threonine (T)
2) Aspartic acid (D) and Glutamic acid (E)
3) Asparagine (N) and Glutamine (Q)
4) Arginine (R) and Lysine (K)
5) Isoleucine (I), Leucine (L), Methionine (M), and Valine (V)
6) Phenylalanine (F), Tyrosine (Y), and Tryptophan (W)

A “non-conservative amino acid substitution” refers to the substitution of a member of one of these classes for a member from another class. In making such changes, according to various embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in various embodiments, the substitution of amino acids whose hydropathic indices are within ±2 is included. In various embodiments, those that are within ±1 are included, and in various embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as disclosed herein. In various embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0.+−.1); glutamate (+3.0.+−.1); serine (+0.3); asparagine (+0.2); glutamine (+0.2); glycine (0); threonine (−0.4); proline (−0.5.+−.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in various embodiments, the substitution of amino acids whose hydrophilicity values are within ±2 is included, in various embodiments, those that are within ±1 are included, and in various embodiments, those within ±0.5 are included.

Exemplary amino acid substitutions are set forth in Table 1.

TABLE 1

| Original Residues | Exemplary Substitutions | Preferred Substitutions |
|---|---|---|
| Ala | Val, Leu, Ile | Val |
| Arg | Lys, Gln, Asn | Lys |
| Asn | Gln | |
| Asp | Glu | |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | |
| Trp | Tyr, Phe | Tyr |
| Tyr | Trp, Phe, Thr, Ser | Phe |
| Val | Ile, Met, Leu, Phe, | Leu |
| | Ala, Norleucine | |

A skilled artisan will be able to determine suitable variants of polypeptides as set forth herein using well-known techniques. In various embodiments, one skilled in the art may identify suitable areas of the molecule that may be changed without destroying activity by targeting regions not believed to be important for activity. In other embodiments, the skilled artisan can identify residues and portions of the molecules that are conserved among similar polypeptides. In further embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, the skilled artisan can predict the importance of amino acid residues in a polypeptide that correspond to amino acid residues important for activity or structure in similar polypeptides. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of a polypeptide with respect to its three-dimensional structure. In various embodiments, one skilled in the art may choose to not make radical changes to amino acid residues predicted to be on the surface of the polypeptide, since such residues may be involved in important interactions with other molecules. Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

The term “polypeptide fragment” and “truncated polypeptide” as used herein refers to a polypeptide that has an amino-terminal and/or carboxy-terminal deletion as compared to a corresponding full-length protein. In various embodiments, fragments can be, e.g., at least 5, at least 10, at least 25, at least 50, at least 100, at least 150, at least 200, at least 250, at least 300, at least 350, at least 400, at least 450, at least 500, at least 600, at least 700, at least 800, at least 900 or at least 1000 amino acids in length. In various embodiments, fragments can also be, e.g., at most 1000, at most 900, at most 800, at most 700, at most 600, at most 500, at most 450, at most 400, at most 350, at most 300, at most 250, at most 200, at most 150, at most 100, at most 50, at most 25, at most 10, or at most 5 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence).

The terms “polypeptide variant”, “hybrid polypeptide” and “polypeptide mutant” as used herein refers to a polypeptide that comprises an amino acid sequence wherein one or more amino acid residues are inserted into, deleted from and/or substituted into the amino acid sequence relative to another polypeptide sequence. In various embodiments, the number of amino acid residues to be inserted, deleted, or substituted can be, e.g., at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length. Hybrids of the present disclosure include fusion proteins.

As described herein, a single mutation will be identified by the particular amino acid substitution at a specific amino acid position within the sequence of a wild-type IFN. For example, for the human IFN-α2b provided as SEQ ID NO: 6, a mutation comprising a tyrosine substituted for the full-length wild-type histidine at amino acid 57 is identified as H57Y.

A “derivative” of a polypeptide is a polypeptide that has been chemically modified, e.g., conjugation to another chemical moiety such as, for example, polyethylene glycol, albumin (e.g., human serum albumin), phosphorylation, and glycosylation.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm and means that a given sequence is at least 80% identical to another length of another sequence. In various embodiments, the % identity is selected from, e.g., at least 60%, at least 65%, at least 70%, at least 75%, at least 80%, at least 85%, at least 90%, at least 95%, or at least 99% or more sequence identity to a given sequence. In various embodiments, the % identity is in the range of, e.g., about 60% to about 70%, about 70% to about 80%, about 80% to about 85%, about 85% to about 90%, about 90% to about 95%, or about 95% to about 99%.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. In various embodiments, the % homology is selected from, e.g., at least 60%, at least 65%, at least 70%, at least 75%, at least 80%, at least 85%, at least 90%, at least 95%, or at least 99% or more sequence homology to a given sequence. In various embodiments, the % homology is in the range of, e.g., about 60% to about 70%, about 70% to about 80%, about 80% to about 85%, about 85% to about 90%, about 90% to about 95%, or about 95% to about 99%.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., J. Mol. Biol. 215:403-10, 1990 (with special reference to the published default setting, i.e., parameters w=4, t=17) and Altschul et al., Nucleic Acids Res., 25:3389-3402, 1997. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix.

In addition to calculating percent sequence identity, the BLAST algorithm also performs a statistical analysis of the similarity between two sequences (see, e.g., Karlin & Altschul, Proc. Nat'l. Acad. Sci. USA, 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is, e.g., less than about 0.1, less than about 0.01, or less than about 0.001.

The term “modification” as used herein refers to any manipulation of the peptide backbone (e.g., amino acid sequence) or the post-translational modifications (e.g., glycosylation) of a polypeptide.

The term “therapeutic protein” refers to proteins, polypeptides, antibodies, peptides or fragments or variants thereof, having one or more therapeutic and/or biological activities. Therapeutic proteins encompassed by the invention include but are not limited to, proteins, polypeptides, peptides, antibodies, and biologics (the terms peptides, proteins, and polypeptides are used interchangeably herein). It is specifically contemplated that the term “therapeutic protein” encompasses the fusion molecules of the present invention.

The term “fusion protein” as used herein refers to a fusion polypeptide molecule comprising two or more genes that originally coded for separate proteins, wherein the components of the fusion protein are linked to each other by peptide-bonds, either directly or through peptide linkers. The term “fused” as used herein refers to components that are linked by peptide bonds, either directly or via one or more peptide linkers.

“Linker” refers to a molecule that joins two other molecules, either covalently, or through ionic, van der Waals or hydrogen bonds, e.g., a nucleic acid molecule that hybridizes to one complementary sequence at the 5′ end and to another complementary sequence at the 3′ end, thus joining two non-complementary sequences. A “cleavable linker” refers to a linker that can be degraded or otherwise severed to separate the two components connected by the cleavable linker. Cleavable linkers are generally cleaved by enzymes, typically peptidases, proteases, nucleases, lipases, and the like. Cleavable linkers may also be cleaved by environmental cues, such as, for example, changes in temperature, pH, salt concentration, etc.

The term “peptide linker” as used herein refers to a peptide comprising one or more amino acids, typically about 2-20 amino acids. Peptide linkers are known in the art or are described herein. Suitable, non-immunogenic linker peptides include, for example, (G₄S)ₙ, (SG₄)ₙ or G₄(SG₄) n peptide linkers. “n” is generally a number between 1 and 10, typically between 2 and 4.

As used herein, cancer refers to any uncontrolled growth of cells that may interfere with the normal functioning of the bodily organs and systems, and includes both primary and metastatic tumors. Primary tumors or cancers that migrate from their original location and seed vital organs can eventually lead to the death of the subject through the functional deterioration of the affected organs. A metastasis is a cancer cell or group of cancer cells, distinct from the primary tumor location, resulting from the dissemination of cancer cells from the primary tumor to other parts of the body. Metastases may eventually result in death of a subject. For example, cancers can include benign and malignant cancers, polyps, hyperplasia, as well as dormant tumors or micrometastases.

The term “tumor associated antigen” (TAA) refers to, e.g., cell surface antigens that are selectively expressed by cancer cells or over-expressed in cancer cells relative to most normal cells. The terms “TAA variant” and “TAA mutant” as used herein refers to a TAA that comprises an amino acid sequence wherein one or more amino acid residues are inserted into, deleted from and/or substituted into the amino acid sequence relative to another TAA sequence. In various embodiments, the number of amino acid residues to be inserted, deleted, or substituted can be, e.g., at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length.

As used herein, the term “tumor microenvironment” refers to the cellular environment in which the tumor exists, including surrounding blood vessels, immune cells, fibroblasts, bone marrow-derived inflammatory cells, lymphocytes, signaling molecules and the extracellular matrix (ECM). Components in the tumor microenvironment can modulate the growth of tumor cells, e.g., their ability to progress and metastasize. The tumor microenvironment can also be influenced by the tumor releasing extracellular signals, promoting tumor angiogenesis and inducing peripheral immune tolerance.

“Pharmaceutical composition” refers to a composition suitable for pharmaceutical use in an animal. A pharmaceutical composition comprises a pharmacologically effective amount of an active agent and a pharmaceutically acceptable carrier. “Pharmacologically effective amount” refers to that amount of an agent effective to produce the intended pharmacological result. “Pharmaceutically acceptable carrier” refers to any of the standard pharmaceutical carriers, vehicles, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co, Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

As used herein, “treatment” (and grammatical variations thereof such as “treat” or “treating”) refers to clinical intervention in an attempt to alter the natural course of a disease in the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

The term “effective amount” or “therapeutically effective amount” as used herein refers to an amount of a compound or composition sufficient to treat a specified disorder, condition or disease such as ameliorate, palliate, lessen, and/or delay one or more of its symptoms. In reference to NHL and other cancers or other unwanted cell proliferation, an effective amount comprises an amount sufficient to: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. An effective amount can be administered in one or more administrations.

“Adjuvant setting” refers to a clinical setting in which an individual has had a history of a proliferative disease, particularly cancer, and generally (but not necessarily) been responsive to therapy, which includes, but is not limited to, surgery (such as surgical resection), radiotherapy, and chemotherapy. However, because of their history of the proliferative disease (such as cancer), these individuals are considered at risk of development of the disease. Treatment or administration in the “adjuvant setting” refers to a subsequent mode of treatment. The degree of risk (i.e., when an individual in the adjuvant setting is considered as “high risk” or “low risk”) depends upon several factors, most usually the extent of disease when first treated.

The phrase “administering” or “cause to be administered” refers to the actions taken by a medical professional (e.g., a physician), or a person controlling medical care of a patient, that control and/or permit the administration of the agent(s)/compound(s) at issue to the patient. Causing to be administered can involve diagnosis and/or determination of an appropriate therapeutic regimen, and/or prescribing particular agent(s)/compounds for a patient. Such prescribing can include, for example, drafting a prescription form, annotating a medical record, and the like. Where administration is described herein, “causing to be administered” is also contemplated.

As used herein, the terms “co-administration”, “co-administered” and “in combination with”, referring to the fusion molecules of the invention and one or more other therapeutic agents, is intended to mean, and does refer to and include the following: simultaneous administration of such combination of fusion molecules of the invention and therapeutic agent(s) to an individual in need of treatment, when such components are formulated together into a single dosage form which releases said components at substantially the same time to said individual; substantially simultaneous administration of such combination of fusion molecules of the invention and therapeutic agent(s) to an individual in need of treatment, when such components are formulated apart from each other into separate dosage forms which are taken at substantially the same time by said individual, whereupon said components are released at substantially the same time to said individual; sequential administration of such combination of fusion molecules of the invention and therapeutic agent(s) to an individual in need of treatment, when such components are formulated apart from each other into separate dosage forms which are taken at consecutive times by said individual with a significant time interval between each administration, whereupon said components are released at substantially different times to said individual; and sequential administration of such combination of fusion molecules of the invention and therapeutic agent(s) to an individual in need of treatment, when such components are formulated together into a single dosage form which releases said components in a controlled manner whereupon they are concurrently, consecutively, and/or overlappingly released at the same and/or different times to said individual, where each part may be administered by either the same or a different route.

The terms “patient,” “individual,” and “subject” may be used interchangeably and refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In various embodiments, the patient can be a human (e.g., adult male, adult female, adolescent male, adolescent female, male child, female child) under the care of a physician or other health worker in a hospital, psychiatric care facility, as an outpatient, or other clinical context. In various embodiments, the patient may be an immunocompromised patient or a patient with a weakened immune system including, but not limited to patients having primary immune deficiency, AIDS; cancer and transplant patients who are taking certain immunosuppressive drugs; and those with inherited diseases that affect the immune system (e.g., congenital agammaglobulinemia, congenital IgA deficiency). In various embodiments, the patient has an immunogenic cancer, including, but not limited to bladder cancer, lung cancer, melanoma, and other cancers reported to have a high rate of mutations (Lawrence et al., Nature, 499 (7457): 214-218, 2013).

The term “immunotherapy” refers to cancer treatments which include, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, OX-40, CD137, GITR, LAG3, TIM-3, SIRP, CD40, CD47, Siglec 8, Siglec 9, Siglec 15, TIGIT and VISTA; treatment using bispecific T cell engaging antibodies (BiTE®) such as blinatumomab: treatment involving administration of biological response modifiers such as IL-2, IL-12, IL-15, IL-21, GM-CSF, IFN-α, IFN-β and IFN-γ; treatment using therapeutic vaccines such as sipuleucel-T; treatment using Bacilli Calmette-Guerin (BCG); treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod.

“Resistant or refractory cancer” refers to tumor cells or cancer that do not respond to previous anti-cancer therapy including, e.g., chemotherapy, surgery, radiation therapy, stem cell transplantation, and immunotherapy. Tumor cells can be resistant or refractory at the beginning of treatment, or they may become resistant or refractory during treatment. Refractory tumor cells include tumors that do not respond at the onset of treatment or respond initially for a short period but fail to respond to treatment. Refractory tumor cells also include tumors that respond to treatment with anticancer therapy but fail to respond to subsequent rounds of therapies. For purposes of this invention, refractory tumor cells also encompass tumors that appear to be inhibited by treatment with anticancer therapy but recur up to five years, sometimes up to ten years or longer after treatment is discontinued. The anticancer therapy can employ chemotherapeutic agents alone, radiation alone, targeted therapy alone, immunotherapy alone, surgery alone, or combinations thereof. For ease of description and not limitation, it will be understood that the refractory tumor cells are interchangeable with resistant tumor.

As used herein, “specific binding” is meant that the binding is selective for the antigen and can be discriminated from unwanted or non-specific interactions. The ability of an immunoglobulin to bind to a specific antigen can be measured either through an enzyme-linked immunosorbent assay (ELISA) or other techniques familiar to one of skill in the art, e.g., Surface Plasmon Resonance (SPR) technique.

The terms “affinity” or “binding affinity” as used herein refers to the strength of the sum total of non-covalent interactions between a single binding site of a molecule (e.g. an antibody) and its binding partner (e.g. an antigen). The affinity of a molecule X for its partner Y can generally be represented by the dissociation constant (KD), which is the ratio of dissociation and association rate constants (koff and kon, respectively). A particular method for measuring affinity is Surface Plasmon Resonance (SPR).

The term “reduced binding”, as used herein refers to a decrease in affinity for the respective interaction, as measured for example by SPR. Conversely, “increased binding” refers to an increase in binding affinity for the respective interaction.

The term “polymer” as used herein generally includes, but is not limited to, homopolymers; copolymers, such as, for example, block, graft, random and alternating copolymers; and terpolymers; and blends and modifications thereof. Furthermore, unless otherwise specifically limited, the term “polymer” shall include all possible geometrical configurations of the material. These configurations include, but are not limited to isotactic, syndiotactic, and random symmetries.

“Polynucleotide” refers to a polymer composed of nucleotide units. Polynucleotides include naturally occurring nucleic acids, such as deoxyribonucleic acid (“DNA”) and ribonucleic acid (“RNA”) as well as nucleic acid analogs. Nucleic acid analogs include those which include non-naturally occurring bases, nucleotides that engage in linkages with other nucleotides other than the naturally occurring phosphodiester bond or which include bases attached through linkages other than phosphodiester bonds. Thus, nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like. Such polynucleotides can be synthesized, for example, using an automated DNA synthesizer. The term “nucleic acid” typically refers to large polynucleotides. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand having the same sequence as an mRNA transcribed from that DNA and which are located 5′ to the 5′-end of the RNA transcript are referred to as “upstream sequences”; sequences on the DNA strand having the same sequence as the RNA and which are 3′ to the 3′ end of the coding RNA transcript are referred to as “downstream sequences.”

A “vector” is a polynucleotide that can be used to introduce another nucleic acid linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), wherein additional DNA segments can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide.

A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06. A nucleotide sequence is “operably linked” to a regulatory sequence if the regulatory sequence affects the expression (e.g., the level, timing, or location of expression) of the nucleotide sequence.

A “host cell” is a cell that can be used to express a polynucleotide of the disclosure. A host cell can be a prokaryote, for example, E. coli, or it can be a eukaryote, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Typically, a host cell is a cultured cell that can be transformed or transfected with a polypeptide-encoding nucleic acid, which can then be expressed in the host cell. The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

The term “isolated molecule” (where the molecule is, for example, a polypeptide or a polynucleotide) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

A protein or polypeptide is “substantially pure,” “substantially homogeneous,” or “substantially purified” when at least about 60% to 75% of a sample exhibits a single species of polypeptide. The polypeptide or protein may be monomeric or multimeric. A substantially pure polypeptide or protein will typically comprise about 50%, 60%, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term “heterologous” as used herein refers to a composition or state that is not native or naturally found, for example, that may be achieved by replacing an existing natural composition or state with one that is derived from another source. Similarly, the expression of a protein in an organism other than the organism in which that protein is naturally expressed constitutes a heterologous expression system and a heterologous protein.

As used herein and in the appended claims, the singular forms “a,” “or,” and “the” include plural referents unless the context clearly dictates otherwise. It is understood that aspect and embodiments of the disclosure described herein include “consisting” and/or “consisting essentially of” aspects and embodiments.

Reference to “about” a value or parameter herein includes (and describes) variations that are directed to that value or parameter per se. For example, description referring to “about X” includes description of “X”.

### Interferon and Interferon Mutants

In the fusion molecules of the present disclosure, either the N- or C-terminus of a TAA antibody, or antigen-binding fragment heavy or light chain will be genetically constructed with one of the several contemplated interferons or interferon mutants. Interferons include type I interferons (e.g., IFN-α, IFN-β) as well as type II interferons (e.g., IFN-γ). The term “interferon” as used herein refers to a full-length interferon or to an interferon fragment (truncated interferon) or to an interferon mutant (truncated interferon and interferon mutant collectively referred to herein as ‘modified interferon’), that substantially retains the biological activity of the full length wild-type interferon (e.g., retains at least 50%, for example at least about any of 60%, 70%, 80%, 90%, or more biological activity of the full length wild-type interferon), including any biosimilar, biogeneric, follow-on biologic, or follow-on protein version of an interferon taught in the art. The interferon can be from essentially any mammalian species. In various embodiments, the interferon is from a species selected from the group consisting of human, equine, bovine, rodent, porcine, lagomorph, feline, canine, murine, caprine, ovine, a non-human primate, and the like. Various such interferons have been extensively described in the literature and are well known to one of ordinary skill in the art (see, e.g., Pestka, Immunological Reviews, 202(1):8-32, 2004). FDA-approved interferons include, e.g., ROFERON®-A (Roche), INTRON® A (Schering), AVONEX® (Biogen, Inc.), BETASERON® (Chiron Corporation) and REBIF® (EMD Serono and Pfizer).

Consensus interferon (con-IFN-α) (also referred to as “IFN-alfacon-1”), a non-natural recombinant interferon, is a second-generation cytokine that was engineered to contain the most frequently occurring amino acids among the non-allelic IFN-α subtypes in humans to form a consensus molecule (Blatt et al., J Interferon Cytokine Res., 16:489-99, 1996). Con-IFN-α shows a higher biological and antiviral capacity in vitro than the non-allelic IFN-α subtypes and the FDA-approved INFERGEN® (InterMune, Inc) has been used internationally to treat patients with chronic hepatitis C (HCV) infection.

In various embodiments, the TAA antibody-IFN fusion molecules comprise an interferon or a modified interferon that possesses, e.g., at least 10%, at least 20%, at least 30%, at least 40%, at least 50%, at least 55%, at least 60%, at least 65%, at least 70%, at least 75%, at least 80%, at least 85%, at least 90%, at least 95%, at least 96%, at least 97%, at least 98%, at least 99%, at least 100%, of the endogenous activity of the wild-type interferon having the same amino acid sequence but not attached to an antibody.

In various embodiments, the TAA antibody-IFN fusion molecules will comprise an interferon or a modified interferon that possesses, e.g., less than 10%, less than 20%, less than 30%, less than 40%, less than 50%, less than 55%, less than 60%, less than 65%, less than 70%, less than 75%, less than 80%, less than 85%, less than 90%, less than 95%, less than 96%, less than 97%, less than 98%, less than 99%, less than 100%, of the endogenous activity of the wild-type interferon having the same amino acid sequence but not attached to an antibody.

Interferon activity can be assessed, for example, using the various anti-viral and anti-proliferative assays described in art (see, e.g., U.S. Pat. No. 8,563,692, U.S. Pat. Public. No. 20130230517, U.S. Pat. Public. No. 20110158905, PCT WO/2014/028502, and PCT WO/2013/059885) as well as the assays described in the Examples section below.

In various embodiments, the TAA antibody-IFN fusion molecules will show at least 10, at least 100, at least 1000, at least 10,000, or at least 100,000-fold selectivity toward cells that express the TAA to which the antibody binds over cells that do not express the TAA, when compared to interferon having the same amino acid sequence not attached to an antibody.

In various embodiments use of a mutated con-IFN-α is contemplated. Single point mutations contemplated for use herein include, but are not limited to, a series of mostly single point mutations in amino acid residues that are considered important to the binding affinity of IFN-α to IFN-αR1 based on published information on NMR structure with the assumption that a single point mutation may change the binding affinity but will not completely knock off the activity of con-IFN-α, therefore still retaining the anti-proliferative properties albeit at much higher concentrations. This will potentially improve the therapeutic index of the fusion molecules comprising an antibody fused to the interferon-alpha mutants. As described herein, a single mutation will be identified by the particular amino acid substitution at a specific amino acid position within the sequence of con-IFN-α provided as SEQ ID NO: 1. For example, a mutation comprising an alanine substituted for the full-length wild-type arginine at amino acid 150 is identified as R150A.

In various embodiments of the present disclosure, the TAA antibody-IFN fusion molecule comprises an interferon mutant comprising one or more amino acid substitutions, insertions, and/or deletions. Means of identifying such modified interferon molecules are routine to those of skill in the art. In one illustrative approach, a library of truncated and/or mutated IFN-α is produced and screened for IFN-α activity. Methods of producing libraries of polypeptide variants are well known to those of skill in the art. The resultant library members can then be screened according to standard methods know to those of skill in the art. Thus, for example, IFN-α activity can be assayed by measuring antiviral activity against a particular test virus. Kits for assaying for IFN-α activity are commercially available (see, e.g., ILITE™ alphabeta kit by Neutekbio, Ireland).

In various embodiments, the interferon contains an amino acid sequence that shares an observed homology of, e.g., at least 70%, at least 75%, at least 80%, at least 85%, at least 90%, at least 95%, at least 96%, at least 97%, at least 98%, or at least 99% with the wildtype con-IFN-α sequence provided below as SEQ ID NO: 1. In some embodiments, the interferon has less than any of about 70%, 75%, 80%, 85%, 90%, or 95%, activity of con-IFN-α provided below as SEQ ID NO: 1:

(SEQ ID NO: 1)
CDLPQTHSLGNRRALILLAQMRRISPFSCLKDRHDFGFPQEEFDGNQFQ
KAQAISVLHEMIQQTFNLFSTKDSSAAWDESLLEKFYTELYQQLNDLEA
CVIQEVGVEETPLMNVDSILAVKKYFQRITLYLTEKKYSPCAWEVVRAE
IMRSFSLSTNLQERLRRKE

In various embodiments, the interferon is a con-IFN-α mutant molecule wherein the arginine at amino acid residue 150 of SEQ ID NO: 1 is replaced with an alanine (R150A). This con-IFN-α mutant molecule is referred to hereinafter as “con-IFN-α (R150A)”. The amino acid sequence of con-IFN-α (R150A) is provided below as SEQ ID NO: 2:

(SEQ ID NO: 2)
CDLPQTHSLGNRRALILLAQMRRISPFSCLKDRHDFGFPQEEFDG
NQFQKAQAISVLHEMIQQTFNLFSTKDSSAAWDESLLEKFYTELY
QQLNDLEACVIQEVGVEETPLMNVDSILAVKKYFQRITLYLTEKK
YSPCAWEVVRAEIMASFSLSTNLQERLRRKE

In various embodiments, the interferon is a con-IFN-α mutant molecule wherein the alanine at amino acid residue 146 of SEQ ID NO: 1 is replaced with an aspartic acid (A146D). This con-IFN-α mutant molecule is referred to hereinafter as “con-IFN-α (A146D)”. The amino acid sequence of con-IFN-α (A146D) is provided below as SEQ ID NO: 3.

(SEQ ID NO: 3)
CDLPQTHSLGNRRALILLAQMRRISPFSCLKDRHDFGFPQEEFDG
NQFQKAQAISVLHEMIQQTFNLFSTKDSSAAWDESLLEKFYTELY
QQLNDLEACVIQEVGVEETPLMNVDSILAVKKYFQRITLYLTEKK
YSPCAWEVVRDEIMRSFSLSTNLQERLRRKE

In various embodiments, the interferon is a con-IFN-α mutant molecule wherein the alanine at amino acid residue 146 of SEQ ID NO: 1 is replaced with a lysine (A146K). This con-IFN-α mutant molecule is referred to hereinafter as “con-IFN-α (A146K)”. The amino acid sequence of con-IFN-α (A146K) is provided below as SEQ ID NO: 4:

(SEQ ID NO: 4)
CDLPQTHSLGNRRALILLAQMRRISPFSCLKDRHDFGFPQEEFDG
NQFQKAQAISVLHEMIQQTFNLFSTKDSSAAWDESLLEKFYTELY
QQLNDLEACVIQEVGVEETPLMNVDSILAVKKYFQRITLYLTEKK
YSPCAWEVVRKEIMRSFSLSTNLQERLRRKE

In various embodiments, the interferon is a con-IFN-α mutant molecule wherein the alanine at amino acid residue 146 of SEQ ID NO: 1 is replaced with an aspartic acid (A146D) and the arginine at amino acid residue 150 of SEQ ID NO: 1 is replaced with an alanine (R150A). This con-IFN-α mutant molecule is referred to hereinafter as “con-IFN-α (A146D and R150A)”. The amino acid sequence of con-IFN-α (A146D and R150A) is provided below as SEQ ID NO: 5.

(SEQ ID NO: 5)
CDLPQTHSLGNRRALILLAQMRRISPFSCLKDRHDFGFPQEEFDG
NQFQKAQAISVLHEMIQQTFNLFSTKDSSAAWDESLLEKFYTELY
QQLNDLEACVIQEVGVEETPLMNVDSILAVKKYFQRITLYLTEKK
YSPCAWEVVRDEIMASFSLSTNLQERLRRKE

In various embodiments, the interferon contains an amino acid sequence that shares an observed homology of, e.g., at least 70%, at least 75%, at least 80%, at least 85%, at least 90%, at least 95%, at least 96%, at least 97%, at least 98%, or at least 99% with the human IFN-α2b sequence provided below as SEQ ID NO: 6 (hereinafter referred to as “IFN-α2b”). In some embodiments, the interferon has less than any of about 70%, 75%, 80%, 85%, 90%, or 95%, activity of IFN-α2b provided below as SEQ ID NO: 6:

(SEQ ID NO: 6)
CDLPQTHSLGSRRTLMLLAQMRRISLFSCLKDRHDFGFPQEEFGN
QFQKAETIPVLHEMIQQIFNLFSTKDSSAAWDETLLDKFYTELYQ
QLNDLEACVIQGVGVTETPLMKEDSILAVRKYFQRITLYLKEKKY
SPCAWEVVRAEIMRSFSLSTNLQESLRSKE

In various embodiments, the interferon is a IFN-α2b mutant molecule wherein the arginine at amino acid residue 149 of SEQ ID NO: 6 is replaced with an alanine (R149A). This IFN-α2b mutant molecule is referred to hereinafter as “IFN-α2b (R149A)”. The amino acid sequence of IFN-α2b (R149A) is provided below as SEQ ID NO: 7:

(SEQ ID NO: 7)
CDLPQTHSLGSRRTLMLLAQMRRISLFSCLKDRHDFGFPQEEFGN
QFQKAETIPVLHEMIQQIFNLFSTKDSSAAWDETLLDKFYTELYQ
QLNDLEACVIQGVGVTETPLMKEDSILAVRKYFQRITLYLKEKKY
SPCAWEVVRAEIMASFSLSTNLQESLRSKE

In various embodiments, the interferon contains an amino acid sequence that shares an observed homology of, e.g., at least 70%, at least 75%, at least 80%, at least 85%, at least 90%, at least 95%, at least 96%, at least 97%, at least 98%, or at least 99% with the human IFN-α5 sequence provided below as SEQ ID NO: 8 (hereinafter referred to as “IFN-α5”). In some embodiments, the interferon has less than any of about 70%, 75%, 80%, 85%, 90%, or 95%, activity of IFN-α5 provided below as SEQ ID NO: 8:

(SEQ ID NO: 8)
CDLPQTHSLSNRRTLMIMAQMGRISPFSCLKDRHDFGFPQEEFDG
NQFQKAQAISVLHEMIQQTFNLFSTKDSSATWDETLLDKFYTELY
QQLNDLEACMMQEVGVEDTPLMNVDSILTVRKYFQRITLYLTEKK
YSPCAWEVVRAEIMRSFSLSANLQERLRRKE

In various embodiments, the interferon is a IFN-α5 mutant molecule wherein the arginine at amino acid residue 150 of SEQ ID NO: 8 is replaced with an alanine (R150A). This IFN-α5 mutant molecule is referred to hereinafter as “IFN-α5 (R150A)”. The amino acid sequence of IFN-α5 (R150A) is provided below as SEQ ID NO: 9.

(SEQ ID NO: 9)
CDLPQTHSLSNRRTLMIMAQMGRISPFSCLKDRHDFGFPQEEFDG
NQFQKAQAISVLHEMIQQTFNLFSTKDSSATWDETLLDKFYTELY
QQLNDLEACMMQEVGVEDTPLMNVDSILTVRKYFQRITLYLTEKK
YSPCAWEVVRAEIMASFSLSANLQERLRRKE

In various embodiments, the interferon is a IFN-α5 mutant molecule wherein the alanine at amino acid residue 146 of SEQ ID NO: 8 is replaced with an aspartic acid (A146D). This IFN-α5 mutant molecule is referred to hereinafter as “IFN-α5 (A146D)”. The amino acid sequence of IFN-α5 (A146D) is provided below as SEQ ID NO: 10:

(SEQ ID NO: 10)
CDLPQTHSLSNRRTLMIMAQMGRISPFSCLKDRHDFGFPQEEFDG
NQFQKAQAISVLHEMIQQTFNLFSTKDSSATWDETLLDKFYTELY
QQLNDLEACMMQEVGVEDTPLMNVDSILTVRKYFQRITLYLTEKK
YSPCAWEVVRDEIMRSFSLSANLQERLRRKE

In various embodiments, the interferon contains an amino acid sequence that shares an observed homology of, e.g., at least 70%, at least 75%, at least 80%, at least 85%, at least 90%, at least 95%, at least 96%, at least 97%, at least 98%, or at least 99% with the human IFN-α6 sequence provided below as SEQ ID NO: 11 (hereinafter referred to as “IFN-α6”). In some embodiments, the interferon has less than any of about 70%, 75%, 80%, 85%, 90%, or 95%, activity of IFN-6 provided below as SEQ ID NO: 11:

(SEQ ID NO: 11)
CDLPQTHSLGHRRTMMLLAQMRRISLFSCLKDRHDFRFPQEEFDG
NQFQKAEAISVLHEVIQQTFNLFSTKDSSVAWDERLLDKLYTELY
QQLNDLEACVMQEVWVGGTPLMNEDSILAVRKYFQRITLYLTEKK
YSPCAWEVVRAEIMRSFSSSRNLQERLRRKE

In various embodiments, the interferon is a IFN-α6 mutant molecule wherein the arginine at amino acid residue 150 of SEQ ID NO: 11 is replaced with an alanine (R150A). This IFN-α6 mutant molecule is referred to hereinafter as “IFN-α6 (R150A)”. The amino acid sequence of IFN-α6 (R150A) is provided below as SEQ ID NO: 12.

(SEQ ID NO: 12)
CDLPQTHSLGHRRTMMLLAQMRRISLFSCLKDRHDFRFPQEEFDG
NQFQKAEAISVLHEVIQQTFNLFSTKDSSVAWDERLLDKLYTELY
QQLNDLEACVMQEVWVGGTPLMNEDSILAVRKYFQRITLYLTEKK
YSPCAWEVVRAEIMASFSSSRNLQERLRRKE

In various embodiments, the interferon is a IFN-α6 mutant molecule wherein the alanine at amino acid residue 146 of SEQ ID NO: 11 is replaced with an aspartic acid (A146D). This IFN-α6 mutant molecule is referred to hereinafter as “IFN-α6 (A146D)”. The amino acid sequence of IFN-α6 (A146D) is provided below as SEQ ID NO: 13:

(SEQ ID NO: 13)
CDLPQTHSLGHRRTMMLLAQMRRISLFSCLKDRHDFRFPQEEFDG
NQFQKAEAISVLHEVIQQTFNLFSTKDSSVAWDERLLDKLYTELY
QQLNDLEACVMQEVWVGGTPLMNEDSILAVRKYFQRITLYLTEKK
YSPCAWEVVRDEIMRSFSSSRNLQERLRRKE

In various embodiments, the interferon is a IFN-α6 mutant molecule wherein the alanine at amino acid residue 146 of SEQ ID NO: 11 is replaced with a lysine (A146K). This IFN-α6 mutant molecule is referred to hereinafter as “IFN-α6 (A146K)”. The amino acid sequence of IFN-α6 (A146K) is provided below as SEQ ID NO: 14:

(SEQ ID NO: 14)
CDLPQTHSLGHRRTMMLLAQMRRISLFSCLKDRHDFRFPQEEFDG
NQFQKAEAISVLHEVIQQTFNLFSTKDSSVAWDERLLDKLYTELY
QQLNDLEACVMQEVWVGGTPLMNEDSILAVRKYFQRITLYLTEKK
YSPCAWEVVRKEIMRSFSSSRNLQERLRRKE

In various embodiments, the interferon is an IFN-α2b mutant molecule having the amino acid sequence set forth in SEQ ID NO: 6, and comprising one or more single point mutations selected from L15A, A19W, R22A, R23A, S25A, L26A, F27A, L30A, L30V, K31A, D32A, R33K, R33A, R33Q, H34A, D35E, Q40A, H57A, H57S, H57Y, E58A, E58N, E58S, Q61A, Q61S, D114R, L117A, R120A, R125A, R125E, K131A, E132A, K133A, K134A, R144A, R144D, R144E, R144G, R144H, R1441, R144K, R144L, R144N, R144Q, R144S, R144T, R144V, R144Y, A145D, A145E, A145G, A145H, A1451, A145K, A145L, A145M, A145N, A145Q, A145R, A145S, A145T, A145V, A145Y, M148A, R149A, S152A, L153A, N156A, R162A, or E165D.

In various embodiments, the interferon is an con-IFN-α mutant molecule having the amino acid sequence set forth in SEQ ID NO: 1, and comprising one or more single point mutations selected from L15A, A19W, R22A, R23A, S25A, L26A, F27A, L30A, L30V, K31A, D32A, R33K, R33A, R33Q, H34A, D35E, Q40A, H58A, H58S, H58Y, E59A, E59N, E59S, Q62A, Q62S, D115R, L118A, R121A, R126A, R126E, K132A, E133A, K134A, K135A, R145A, R145D, R145E, R145G, R145H, R1451, R145K, R145L, R145N, R145Q, R145S, R145T, R145V, R145Y, A146D, A146E, A146G, A146H, A1461, A146K, A146L, A146M, A146N, A146Q, A146R, A146S, A146T, A146V, A146Y, M149A, R150A, S153A, L154A, N157A, R163A, or E166D.

Additional interferon mutants contemplated for use include those described in, e.g., U.S. Pat. No. 9,611,322 (Wilson et al.), U.S. Pat. No. 8,258,263 (Morrison et al), U.S. Pat. No. 9,492,562 (Tavernier et al.), U.S. Pat. No. 10,259,854 (Grewal et al), each of which is hereby incorporated by reference in its entirety for the interferon mutants and sequences provided therein. In various embodiments, the interferon contains an amino acid sequence that shares an observed homology of, e.g., at least 70%, at least 75%, at least 80%, at least 85%, at least 90%, at least 95%, at least 96%, at least 97%, at least 98%, or at least 99% with the wildtype IFN-α sequence selected from the group consisting of IFN-α5 (NP_002160.1), IFN-α6 (NP_066282.1), IFN-α7 (NP_066401.1), IFN-α8 (NP_002161.2), IFN-10 (NP_002162.1), IFN-α16 (NP_002164.1), IFN-α17 (NP_067091.1), IFN-α21 (NP_002166.2), and con-IFN-α (DB00069).

### Targeting Moieties—Tumor Associated Antigen Antibodies

The methods of the present invention utilize isolated non-occurring genetically engineered bifunctional fusion molecules comprising at least one interferon, or interferon mutant molecule attached to at least one targeting moieties having recognition domains which specifically bind to a target (e.g. antigen, receptor) of interest. In various embodiments, the target (e.g. antigen, receptor) of interest can be found on one or more immune cells, which can include, without limitation, T cells, cytotoxic T lymphocytes, T helper cells, natural killer (NK) cells, natural killer T (NKT) cells, anti-tumor macrophages (e.g. M1 macrophages), B cells, dendritic cells, or subsets thereof. In various embodiments, the recognition domains specifically bind to a target (e.g. antigen, receptor) of interest and effectively, directly or indirectly, recruit one of more immune cells. In various embodiments, the target (e.g. antigen, receptor) of interest can be found on one or more tumor cells. In some embodiments, the bifunctional fusion proteins comprise two or more targeting moieties. In various embodiments, the bifunctional fusion proteins may directly or indirectly recruit an immune cell, e.g., in some embodiments, to a therapeutic site (e.g. a locus with one or more disease cell or cell to be modulated for a therapeutic effect). In various embodiments, the present bifunctional fusion proteins may directly or indirectly recruit an immune cell, e.g. an immune cell that can kill and/or suppress a tumor cell, to a site of action (such as, by way of non-limiting example, the tumor microenvironment).

In various embodiments, the present bifunctional fusion proteins are capable of, or find use in methods involving, shifting the balance of immune cells in favor of immune attack of a tumor. For instance, the present bifunctional fusion proteins can shift the ratio of immune cells at a site of clinical importance in favor of cells that can kill and/or suppress a tumor (e.g. T cells, cytotoxic T lymphocytes, T helper cells, natural killer (NK) cells, natural killer T (NKT) cells, anti-tumor macrophages (e.g. M1 macrophages), B cells, dendritic cells, or subsets thereof) and in opposition to cells that protect tumors (e.g. myeloid-derived suppressor cells (MDSCs), regulatory T cells (Tregs); tumor associated neutrophils (TANs), M2 macrophages, tumor associated macrophages (TAMs), or subsets thereof). In some embodiments, the present bifunctional fusion protein is capable of increasing a ratio of effector T cells to regulatory T cells.

A wide variety of tumor associated antigens and tumor markers have been described in the literature and are well known to one of ordinary skill in the art. The TAA Ab-IFN fusion molecules used in the methods of the present invention may comprise an antibody, or antigen binding antibody fragment, specific to any of the tumor associated antigens described in the art, including any biosimilar, biogeneric, follow-on biologic, or follow-on protein version of any TAA described in the art. The TAA can be any peptide, polypeptide, protein, nucleic acid, lipid, carbohydrate, or small organic molecule, or any combination thereof, against which the skilled artisan wishes to induce an immune response.

In various embodiments, the TAA contemplated for use includes, but is not limited to those provided in Table 2. Each associated reference is incorporated herein by reference for the purpose of identifying the referenced tumor markers.

TABLE 2
Illustrative Tumor Markers

| Marker | Reference |
|---|---|
| 5 alpha reductase | Delos et al. (1998) Int J Cancer, 75: 6 840-846 |
| a-fetoprotein | Esteban et al. (1996) Tumour Biol., 17(5): 299-305 |
| AM-1 | Harada et al. (1996) Tohoku J Exp Med., 180(3): 273-288 |
| APC | Dihlmannet al. (1997) Oncol Res., 9(3) 119-127 |
| APRIL | Sordat et al. (1998) J Exp Med., 188(6): 1185-1190 |
| BAGE | Boel et al. (1995) Immunity, 2: 167-175. |
| β-catenin | Hugh et al. (1999) Int J Cancer, 82(4): 504-11 |
| Bc12 | Koty et al. (1999) Lung Cancer, 23(2): 115-127 |
| bcr-abl (b3a2) | Verfaillie et al. (1996) Blood, 87(11): 4770-4779 |
| CA-125 (Mucin 16) | Bast et al. (1998) Int J Biol Markers, 13(4): 179-187 |
| CASP-8/FLICE | Mandruzzato et al. (1997)J Exp Med., 186(5): 785-793. |
| Cathepsins | Thomssen et al. (1995) Clin Cancer Res., 1(7): 741-746 |
| CD19 | Scheuermann et al. (1995) Leuk Lymphoma, 18(5-6): 385-397 |
| CD20 | Knox et al. (1996) Clin Cancer Res., 2(3): 457-470 |
| CD21, CD23 | Shubinsky et al. (1997) Leuk Lymphoma, 25(5-6): 521-530 |
| CD22, CD38 | French et al. (1995) Br J Cancer, 71(5): 986-994 |
| CD33 | Nakase et al. (1996) Am J Clin Pathol., 105(6): 761-768 |
| CD35 | Yamakawa et al. Cancer, 73(11): 2808-2817 |
| CD44 | Naot et al. (1997) Adv Cancer Res., 71: 241-319 |
| CD45 | Buzzi et al. (1992) Cancer Res., 52(14): 4027-4035 |
| CD46 | Yamakawa et al. (1994) Cancer, 73(11): 2808-2817 |
| CD5 | Stein et al. (1991) Clin Exp Immunol., 85(3): 418-423 |
| CD52 | Ginaldi et al. (1998) Leuk Res., 22(2): 185-191 |
| CD55 | Spendlove et al. (1999) Cancer Res., 59: 2282-2286. |
| CD59 | Jarvis et al. (1997) Int J Cancer, 71(6): 1049-1055 |
| CDC27 | Wang et al. (1999) Science, 284(5418): 1351-1354 |
| CDK4 | Wolfel et al. (1995) Science, 269(5228): 1281-1284 |
| CEA | Kass et al. (1999) Cancer Res., 59(3): 676-683 |
| c-myc | Watson et al. (1991) Cancer Res., 51(15): 3996-4000 |
| Cox-2 | Tsujii et al. (1998) Cell, 93: 705-716 |
| DCC | Gotley et al. (1996) Oncogene, 13(4): 787-795 |
| DcR3 | Pitti et al. (1998) Nature, 396: 699-703 |
| E6/E7 | SteIler et al. (1996) Cancer Res., 56(21): 5087-5091 |
| EGFR | Yang et al. (1999) Cancer Res., 59(6): 1236-1243. |
| EMBP | Shiina et al. (1996) Prostate, 29(3): 169-176. |
| Ena78 | Arenberg et al. (1998) J. Clin. Invest., 102: 465-472. |
| FGF8b and FGF8a | Dorkin et al. (1999) Oncogene, 18(17): 2755-2761 |
| FLK-1/KDR | Annie and Fong (1999) Cancer Res., 59: 99-106 |
| Folic Acid Receptor | Dixon et al (1992) J Biol Chem., 267(33): 24140-72414 |
| G250 | Divgi et al. (1998) Clin Cancer Res., 4(11): 2729-2739 |
| GAGE-Family | De Backer et al. (1999) Cancer Res., 59(13): 3157-3165 |
| gastrin 17 | Watson et al. (1995) Int J Cancer, 61(2): 233-240 |
| Gastrin-releasing | Wang et al. (1996) Int J Cancer, 68(4): 528-534 |
| hormone (bombesin) | |
| GD2/GD3/GM2 | Wiesner and Sweeley (1995) Int J Cancer, 60(3): 294-299 |
| GnRH | Bahk et al. (1998) Urol Res., 26(4): 259-264 |
| GnTV | Hengstler et al. (1998) Recent Results Cancer Res., 154: 47-85 |
| gp100/Pmel17 | Wagner et al. (1997) Cancer Immunol Immunther. 44(4): 239- 247 |
| gp-100-in4 | Kirkin et al. (1998) APMIS, 106(7): 665-679 |
| gp15 | Maeurer et al. (1996) Melanoma Res., 6(1): 11-24 |
| gp75/TRP-1 | Lewis et al. (1995) Semin Cancer Biol., 6(6): 321-327 |
| hCG | Hoermann et al. (1992) Cancer Res., 52(6): 1520-1524 |
| Heparanase | Vlodavsky et al. (1999) Nat Med., 5(7): 793-802 |
| Her2/neu | Lewis et al. (1995) Semin Cancer Biol., 6(6): 321-327 |
| Her3 | |
| HMTV | Kahl et al. (1991)Br J Cancer, 63(4): 534-540 |
| Hsp70 | Jaattela et al. (1998) EMBO J., 17(21): 6124-6134 |
| hTERT | Vonderheide et al. (1999) Immunity, 10: 673-679. 1999. |
| (telomerase) | |
| IGFR1 | Ellis et al. (1998) Breast Cancer Res. Treat., 52: 175-184 |
| IL-13R | Murata et al. (1997) BiochemBiophysRes Commun., 238(1): 90-94 |
| iNOS | Klotz et al. (1998) Cancer, 82(10): 1897-1903 |
| Ki 67 | Gerdes et al. (1983) Int J Cancer, 31: 13-20 |
| KIAA0205 | Gueguen et al. (1998) J Immunol., 160(12): 6188-6194 |
| K-ras, H-ras, | Abrams et al. (1996) Semin Oncol., 23(1): 118-134 |
| N-ras | |
| KSA | Zhang et al. (1998) Clin Cancer Res., 4(2): 295-302 (CO17-1A) |
| LDLR-FUT | Caruso et al. (1998) Oncol Rep., 5(4): 927-930 |
| MAGE Family | Marchand et al. (1999) Int J Cancer, 80(2): 219-230 |
| (MAGE1, MAGE3, etc.) | |
| Mammaglobin | Watson et al. (1999) Cancer Res., 59: 13 3028-3031 |
| MAP17 | Kocher et al. (1996) Am J Pathol., 149(2): 493-500 |
| Melan-A/ | Lewis and Houghton (1995) Semin Cancer Biol., 6(6): 321-327 |
| MART-1 | |
| mesothelin | Chang et al. (1996)Proc. Natl. Acad. Sci., USA, 93(1): 136-140 |
| MIC A/B | Groh et al. (1998) Science, 279: 1737-1740 |
| MT-MMP's, such as | Sato and Seiki (1996) J Biochem (Tokyo), 119(2): 209-215 |
| MMP2, MMP3, MMP7, | |
| MMP9 | |
| Mox1 | Candia et al. (1992) Development, 116(4): 1123-1136 |
| Mucin, such as MUC-1, | Lewis and Houghton (1995) Semin Cancer Biol., 6(6): 321-327 |
| MUC-2, MUC-3, MUC-4 | |
| MUM-1 | Kirkin et al. (1998) APMIS, 106(7): 665-679 |
| NY-ESO-1 | Jager et al. (1998) J. Exp. Med., 187: 265-270 |
| Osteonectin | Graham et al. (1997) Eur J Cancer, 33(10): 1654-1660 |
| p15 | Yoshida et al. (1995) Cancer Res., 55(13): 2756-2760 |
| P170/MDR1 | Trock et al. (1997) J Natl Cancer Inst., 89(13): 917-931 |
| p53 | Roth et al. (1996) Proc. Natl. Acad. Sci., USA, 93(10): 4781-4786. |
| p97/melanotransferrin | Furukawa et al. (1989) J Exp Med., 169(2): 585-590 |
| PAI-1 | Grondahl-Hansen et al. (1993) Cancer Res., 53(11): 2513-2521 |
| PDGF | Vassbotn et al. (1993) Mol Cell Biol., 13(7): 4066-4076 |
| Plasminogen (uPA) | Naitoh et al. (1995) Jpn J Cancer Res., 86(1): 48-56 |
| PRAME | Kirkin et al. (1998) APMIS, 106(7): 665-679 |
| Probasin | Matuo et al. (1985) BiochemBiophysResComm., 130(1): 293-300 |
| Progenipoietin -- PSA | Sanda et al. (1999) Urology, 53(2): 260-266. |
| PSM | Kawakami et al. (1997) Cancer Res., 57(12): 2321-2324 |
| RAGE-1 | Gaugler et al. (1996) Immunogenetics, 44(5): 323-330 |
| Rb | Dosaka-Akita et al. (1997) Cancer, 79(7): 1329-1337 |
| RCAS1 | Sonoda et al. (1996) Cancer, 77(8): 1501-1509. |
| SART-1 | Kikuchi et al. (1999) Int J Cancer, 81(3): 459-466 |
| SSX gene | Gure et al. (1997) Int J Cancer, 72(6): 965-971 family |
| STAT3 | Bromberg et al. (1999) Cell, 98(3): 295-303 |
| STn | Sandmaier et al. (1999) J Immunother., 22(1): 54-66 |
| (mucin assoc.) | |
| TAG-72 | Kuroki et al. (1990) Cancer Res., 50(16): 4872-4879 |
| TGF-a | Imanishi et al. (1989) Br J Cancer, 59(5): 761-765 |
| TGF-β | Picon et al. (1998) CancerEpidemiolBiomarkerPrey, 7(6): 497-504 |
| Thymosin β 15 | Bao et al. (1996) Nature Medicine. 2(12), 1322-1328 |
| TNF-α | Moradi et al. (1993) Cancer, 72(8): 2433-2440 |
| TPA | Maulard et al. (1994) Cancer, 73(2): 394-398 |
| TPI | Nishida et al. (1984) Cancer Res 44(8): 3324-9 |
| TRP-2 | Parkhurst et al. (1998) Cancer Res., 58(21) 4895-4901 |
| Tyrosinase | Kirkin et al. (1998) APMIS, 106(7): 665-679 |
| VEGF | Hyodo et al. (1998) Eur J Cancer, 34(13): 2041-2045 |
| ZAG | Sanchez et al. (1999) Science, 283(5409): 1914-1919 |
| p16INK4 | Quelle et al. (1995) Oncogene Aug. 17, 1995; 11(4): 635-645 |
| Glutathione | Hengstler (1998) et al. Recent Results Cancer Res., 154: 47-85 |

Any of the foregoing markers can be used as TAAs targets for fusion molecules of this invention. In various embodiments, the one or more TAA, TAA variant, or TAA mutant contemplated for use in the fusion molecule constructs and methods of the present disclosure is selected from, or derived from, the list provided in Table 3.

TABLE 3

| Tumor Associated | |
|---|---|
| Antigen | RefSeq (protein) |
| Her2/neu | NP_001005862 |
| Her3 | NP_001005915 |
| Her4 | NP_001036064 |
| EGF | NP_001171601 |
| EGFR | NP_005219 |
| CD2 | NP_001758 |
| CD3 | NM_000732 |
| CD5 | NP_055022 |
| CD7 | NP_006128 |
| CD13 | NP_001141 |
| CD19 | NP_001171569 |
| CD20 | NP_068769 |
| CD21 | NP_001006659 |
| CD22 | NP_001762 |
| CD23 | NP_001193948 |
| CD30 | NP_001234 |
| CD33 | NP_001234.3 |
| CD34 | NP_001020280 |
| CD38 | NP_001766 |
| CD40 | NP_001241 |
| CD46 | NP_002380 |
| CD55 | NP_000565 |
| CD59 | NP_000602 |
| CD69 | NP_001772 |
| CD70 | NM 001252 |
| CD71 | NP_001121620 |
| CD80 | NP_005182 |
| CD97 | NP_001020331 |
| CD117 | NP_000213 |
| CD127 | NP_002176 |
| CD134 | NP_003318 |
| CD137 | NP_001552 |
| CD138 | NP_001006947 |
| CD146 | NP_006491 |
| CD147 | NP_001719 |
| CD152 | NP_001032720 |
| CD154 | NP_000065 |
| CD195 | NP_000570 |
| CD200 | NP_001004196 |
| CD212 | NP_001276952 |
| CD223 | NP_002277 |
| CD253 | NP_001177871 |
| CD272 | NP_001078826 |
| CD274 | NP_001254635 |
| CD276 | NP_001019907 |
| CD278 | NP_036224 |
| CD279 | NP_005009 |
| CD309 (VEGFR2) | NP_002244 |
| DR6 | NP_055267 |
| PD-L1 | NP_001254635 |
| PD-1 | NP_005009.2 |
| Kv1.3 | NP_002223 |
| 5E10 | NP_006279 |
| MUC1 | NP_001018016 |
| uPA | NM_002658 |
| SLAMF7 (CD319) | NP_001269517 |
| MAGE 3 | NP_005353 |
| MUC 16 (CA-125) | NP_078966 |
| KLK3 | NP_001025218 |
| K-ras | NP_004976 |
| Mesothelin | NP_001170826 |
| p53 | NP_000537 |
| Survivin | NP_001012270 |
| G250 (Renal Cell | GenBank CAB82444.1 |
| Carcinoma Antigen) | |
| PSMA | NP_001014986 |
| HLA-DR | NP_001020330 |
| 1D10 | NP_114143 |
| Collagen Type I | NP_000079 |
| Collagen Type II | NP_000080 |
| Fibronectin | XP_005246463 |
| Tenascin | NP_002151 |
| Fibroblast Activation | NM_004460.3 |
| Protein (FAP) | |
| Matrix Metalloproteinase- | NP_004986 |
| 14 (MMP-14) | |
| Legumain | NP_001008530 |
| Matrix Metalloproteinase-2 | NP_001121363 |
| (MMP-2) | |
| Matrix Metalloproteinase-9 | NP_004985 |
| (MMP-9) | |
| Siglec 8 | NP_055257 |
| Siglec 9 | NP_001185487 |
| Siglec 15 | NP_998767 |
| c-Met | NP_000236.2 |
| CLDN18.2 | NP_001002026.1 |
| CCR1 | NP_001286.1 |
| CCR2 | NP_001116513.2 |
| CCR3 | NP_847899.1 |
| CCR4 | NP_005499.1 |
| CCR5 | AAB57793.1 |
| CCR6 | NP_113597.2 |
| CCR7 | NP_001829.1 |
| CCR8 | NP_005192.1 |
| CCR9 | NP_001373376.1 |
| CCR10 | NP_057686.2 |
| CXCR1 | NP_000625.1 |
| CXCR2 | NP_001161770.1 |
| CXCR3 | NP_001495.1 |
| CXCR4 | P61073-1 |
| CXCR5 | NP_001707.1 |
| CXCR6 | NP_001373364.1 |
| CXCR8 | NP_005292.2 |
| CX3CR1 | NP_001164642.1 |
| XCR1 | NP_001019815.1 |

Methods of generating antibodies that bind to the TAAs described herein are known to those skilled in the art. For example, a method for generating a monoclonal antibody that binds specifically to a targeted antigen polypeptide may comprise administering to a mouse an amount of an immunogenic composition comprising the targeted antigen polypeptide effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monoclonal antibody that binds specifically to the targeted antigen polypeptide. Once obtained, a hybridoma can be propagated in a cell culture, optionally in culture conditions where the hybridoma-derived cells produce the monoclonal antibody that binds specifically to targeted antigen polypeptide. The monoclonal antibody may be purified from the cell culture. A variety of different techniques are then available for testing an antigen/antibody interaction to identify particularly desirable antibodies.

Other suitable methods of producing or isolating antibodies of the requisite specificity can be used, including, for example, methods which select recombinant antibody from a library, or which rely upon immunization of transgenic animals (e.g., mice) capable of producing a full repertoire of human antibodies. See e.g., Jakobovits et al., Proc. Natl. Acad. Sci. (U.S.A.), 90:2551-2555, 1993; Jakobovits et al., Nature, 362:255-258, 1993; Lonberg et al., U.S. Pat. No. 5,545,806; and Surani et al., U.S. Pat. No. 5,545,807.

Antibodies can be engineered in numerous ways. They can be made as bispecific antibodies, heterodimeric antibodies, single-chain antibodies (including small modular immunopharmaceuticals or SMIPs™), Fab and F(ab′)₂ fragments, etc. Antibodies can be humanized, chimerized, deimmunized, or fully human. Numerous publications set forth the many types of antibodies and the methods of engineering such antibodies. For example, see U.S. Pat. Nos. 6,355,245; 6,180,370; 5,693,762; 6,407,213; 6,548,640; 5,565,332; 5,225,539; 6,103,889; and 5,260,203.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al., Science, 240:1041-1043, 1988; Liu et al., Proc. Natl. Acad. Sci. (U.S.A.), 84:3439-3443, 1987; Liu et al., J. Immunol., 139:3521-3526, 1987; Sun et al., Proc. Natl. Acad. Sci. (U.S.A.), 84:214-218, 1987; Nishimura et al., Canc. Res., 47:999-1005, 1987; Wood et al., Nature, 314:446-449, 1985; and Shaw et al., J. Natl Cancer Inst., 80:1553-1559, 1988).

Methods for humanizing antibodies have been described in the art. In some embodiments, a humanized antibody has one or more amino acid residues introduced from a source that is nonhuman, in addition to the nonhuman CDRs. Humanization can be essentially performed following the method of Winter and co-workers (Jones et al., Nature, 321:522-525, 1986; Riechmann et al., Nature, 332:323-327, 1988; Verhoeyen et al., Science, 239:1534-1536, 1988), by substituting hypervariable region sequences for the corresponding sequences of a human antibody. Accordingly, such “humanized” antibodies are chimeric antibodies (U.S. Pat. No. 4,816,567) wherein substantially less than an intact human variable region has been substituted by the corresponding sequence from a nonhuman species. In practice, humanized antibodies are typically human antibodies in which some hypervariable region residues and possibly some framework region residues are substituted by residues from analogous sites in rodent antibodies.

U.S. Pat. No. 5,693,761 to Queen et al, discloses a refinement on Winter et al. for humanizing antibodies, and is based on the premise that ascribes avidity loss to problems in the structural motifs in the humanized framework which, because of steric or other chemical incompatibility, interfere with the folding of the CDRs into the binding-capable conformation found in the mouse antibody. To address this problem, Queen teaches using human framework sequences closely homologous in linear peptide sequence to framework sequences of the mouse antibody to be humanized. Accordingly, the methods of Queen focus on comparing framework sequences between species. Typically, all available human variable region sequences are compared to a particular mouse sequence and the percentage identity between correspondent framework residues is calculated. The human variable region with the highest percentage is selected to provide the framework sequences for the humanizing project. Queen also teaches that it is important to retain in the humanized framework, certain amino acid residues from the mouse framework critical for supporting the CDRs in a binding-capable conformation. Potential criticality is assessed from molecular models. Candidate residues for retention are typically those adjacent in linear sequence to a CDR or physically within 6 Å of any CDR residue.

In other approaches, the importance of particular framework amino acid residues is determined experimentally once a low-avidity humanized construct is obtained, by reversion of single residues to the mouse sequence and assaying antigen binding as described by Riechmann et al, 1988. Another example approach for identifying important amino acids in framework sequences is disclosed by U.S. Pat. No. 5,821,337 to Carter et al, and by U.S. Pat. No. 5,859,205 to Adair et al. These references disclose specific Kabat residue positions in the framework, which, in a humanized antibody may require substitution with the correspondent mouse amino acid to preserve avidity.

Another method of humanizing antibodies, referred to as “framework shuffling”, relies on generating a combinatorial library with nonhuman CDR variable regions fused in frame into a pool of individual human germline frameworks (Dall'Acqua et al., Methods, 36:43, 2005). The libraries are then screened to identify clones that encode humanized antibodies which retain good binding.

The choice of human variable regions, both light and heavy, to be used in making the humanized antibodies is very important to reduce antigenicity. According to the so-called “best-fit” method, the sequence of the variable region of a rodent antibody is screened against the entire library of known human variable-domain sequences. The human sequence that is closest to that of the rodent is then accepted as the human framework region (framework region) for the humanized antibody (Sims et al., J. Immunol., 151:2296, 1993; Chothia et al., J. Mol. Biol., 196:901, 1987). Another method uses a particular framework region derived from the consensus sequence of all human antibodies of a particular subgroup of light or heavy chain variable regions. The same framework may be used for several different humanized antibodies (Carter et al., Proc. Natl. Acad. Sci. (U.S.A.), 89:4285, 1992; Presta et al., J. Immunol., 151:2623, 1993).

The choice of nonhuman residues to substitute into the human variable region can be influenced by a variety of factors. These factors include, for example, the rarity of the amino acid in a particular position, the probability of interaction with either the CDRs or the antigen, and the probability of participating in the interface between the light and heavy chain variable domain interface. (See, for example, U.S. Pat. Nos. 5,693,761, 6,632,927, and 6,639,055). One method to analyze these factors is through the use of three-dimensional models of the non-human and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind its antigen. In this way, nonhuman residues can be selected and substituted for human variable region residues in order to achieve the desired antibody characteristic, such as increased affinity for the target antigen(s).

Methods for making fully human antibodies have been described in the art. By way of example, a method for producing a TAA antibody or antigen-binding fragment thereof comprises the steps of synthesizing a library of human antibodies on phage, screening the library with TAA or an antibody-binding portion thereof, isolating phage that bind TAA, and obtaining the antibody from the phage. By way of another example, one method for preparing the library of antibodies for use in phage display techniques comprises the steps of immunizing a non-human animal comprising human immunoglobulin loci with TAA or an antigenic portion thereof to create an immune response, extracting antibody-producing cells from the immunized animal; isolating RNA encoding heavy and light chains of antibodies of the disclosure from the extracted cells, reverse transcribing the RNA to produce cDNA, amplifying the cDNA using primers, and inserting the cDNA into a phage display vector such that antibodies are expressed on the phage. Recombinant anti-TAA antibodies of the disclosure may be obtained in this way.

Again, by way of example, recombinant human anti-TAA antibodies of the disclosure can also be isolated by screening a recombinant combinatorial antibody library. Preferably the library is a scFv phage display library, generated using human V_{L} and V_{H} CDNAs prepared from mRNA isolated from B cells. Methods for preparing and screening such libraries are known in the art. Kits for generating phage display libraries are commercially available (e.g., the Pharmacia Recombinant Phage Antibody System, catalog no. 27-9400-01; and the Stratagene SurfZAP™ phage display kit, catalog no. 240612). There also are other methods and reagents that can be used in generating and screening antibody display libraries (see, e.g., U.S. Pat. No. 5,223,409; PCT Publication Nos. WO 92/18619, WO 91/17271, WO 92/20791, WO 92/15679, WO 93/01288, WO 92/01047, WO 92/09690; Fuchs et al., Bio/Technology, 9:1370-1372 (1991); Hay et al., Hum. Antibod. Hybridomas, 3:81-85, 1992; Huse et al., Science, 246:1275-1281, 1989; McCafferty et al., Nature, 348:552-554, 1990; Griffiths et al., EMBO J., 12:725-734, 1993; Hawkins et al., J. Mol. Biol., 226:889-896, 1992; Clackson et al., Nature, 352:624-628, 1991; Gram et al., Proc. Natl. Acad. Sci. (U.S.A.), 89:3576-3580, 1992; Garrad et al., Bio/Technology, 9:1373-1377, 1991; Hoogenboom et al., Nuc. Acid Res., 19:4133-4137, 1991; and Barbas et al., Proc. Natl. Acad. Sci. (U.S.A.), 88:7978-7982, 1991), all incorporated herein by reference.

Human antibodies are also produced by immunizing a non-human, transgenic animal comprising within its genome some or all of human immunoglobulin heavy chain and light chain loci with a human IgE antigen, e.g., a XenoMouse™ animal (Abgenix, Inc./Amgen, Inc.—Fremont, Calif.). XenoMouse™ mice are engineered mouse strains that comprise large fragments of human immunoglobulin heavy chain and light chain loci and are deficient in mouse antibody production. See, e.g., Green et al., Nature Genetics, 7:13-21, 1994 and U.S. Pat. Nos. 5,916,771, 5,939,598, 5,985,615, 5,998,209, 6,075,181, 6,091,001, 6,114,598, 6,130,364, 6,162,963 and 6,150,584. XenoMouse™ mice produce an adult-like human repertoire of fully human antibodies and generate antigen-specific human antibodies. In some embodiments, the XenoMouse™ mice contain approximately 80% of the human antibody V gene repertoire through introduction of megabase sized, germline configuration fragments of the human heavy chain loci and kappa light chain loci in yeast artificial chromosome (YAC). In other embodiments, XenoMouse™ mice further contain approximately all of the human lambda light chain locus. See Mendez et al., Nature Genetics, 15:146-156, 1997; Green and Jakobovits, J. Exp. Med., 188:483-495, 1998; and WO 98/24893.

In various embodiments, the fusion molecules of the present disclosure utilize an antibody or antigen-binding fragment thereof that is selected from the group consisting of a polyclonal antibody, a monoclonal antibody, a recombinant antibody, a bispecific antibody, a heterodimeric antibody, a diabody, a chimerized or chimeric antibody or antigen-binding fragment thereof, a humanized antibody or antigen-binding fragment thereof, a fully human antibody or antigen-binding fragment thereof, a CDR-grafted antibody or antigen-binding fragment thereof, a single chain antibody, an Fv, an Fd, an Fab, an Fab′, or an F(ab′)₂, and synthetic or semi-synthetic antibodies.

In various embodiments, the fusion molecules of the present disclosure utilize an antibody or antigen-binding fragment that binds to a TAA with a dissociation constant (K_{D}) of, e.g., at least about 1×10⁻³ M, at least about 1×10⁻⁴ M, at least about 1×10⁻⁵ M, at least about 1×10⁻⁶ M, at least about 1×10⁻⁷ M, at least about 1×10⁻⁸ M, at least about 1×10⁻⁹ M, at least about 1×10⁻¹⁰ M, at least about 1×10⁻¹¹ M, or at least about 1×10⁻¹² M. In various embodiments, the fusion molecules of the present disclosure utilize an antibody or antigen-binding fragment that binds to a TAA with a dissociation constant (K_{D}) in the range of, e.g., at least about 1×10⁻³ M to at least about 1×10⁻⁴ M, at least about 1×10⁻⁴ M to at least about 1×10⁻⁵ M, at least about 1×10⁻⁵ M to at least about 1×10⁻⁶ M, at least about 1×10⁻⁶ M to at least about 1×10⁻⁷ M, at least about 1×10⁻⁷ M to at least about 1×10⁻⁸ M, at least about 1×10⁻⁸ M to at least about 1×10⁻⁹ M, at least about 1×10⁻⁹ M to at least about 1×10⁻¹⁰ M, at least about 1×10⁻¹⁰ M to at least about 1×10⁻¹¹ M, or at least about 1×10⁻¹¹ M to at least about 1×10⁻¹² M.

In various embodiments, the fusion molecules of the present disclosure utilize an antibody or antigen-binding fragment that cross-competes for binding to the same epitope on the TAA as a reference antibody which comprises the heavy chain variable region and light chain variable region set forth in the references and sequence listings provided herein.

A number of immune-checkpoint protein antigens have been reported to be expressed on various immune cells, including, e.g., SIRP (expressed on macrophage, monocytes, dendritic cells), CD47 (highly expressed on tumor cells and other cell types), VISTA (expressed on monocytes, dendritic cells, B cells, T cells), CD152 (expressed by activated CD8+ T cells, CD4+ T cells and regulatory T cells), CD279 (expressed on tumor infiltrating lymphocytes, expressed by activated T cells (both CD4 and CD8), regulatory T cells, activated B cells, activated NK cells, anergic T cells, monocytes, dendritic cells), CD274 (expressed on T cells, B cells, dendritic cells, macrophages, vascular endothelial cells, pancreatic islet cells), and CD223 (expressed by activated T cells, regulatory T cells, anergic T cells, NK cells, NKT cells, and plasmacytoid dendritic cells) (see, e.g., Pardoll, D., Nature Reviews Cancer, 12:252-264, 2012). Antibodies that bind to an antigen which is determined to be an immune-checkpoint protein are known to those skilled in the art. For example, various anti-CD276 antibodies have been described in the art (see, e.g., U.S. Pat. Public. No. 20120294796 (Johnson et al) and references cited therein); various anti-CD272 antibodies have been described in the art (see, e.g., U.S. Pat. Public. No. 20140017255 (Mataraza et al) and references cited therein); various anti-CD152/CTLA-4 antibodies have been described in the art (see, e.g., U.S. Pat. Public. No. 20130136749 (Korman et al) and references cited therein); various anti-LAG-3/CD223 antibodies have been described in the art (see, e.g., U.S. Pat. Public. No. 20110150892 (Thudium et al) and references cited therein); various anti-CD279/PD-1 antibodies have been described in the art (see, e.g., U.S. Pat. No. 7,488,802 (Collins et al) and references cited therein); various anti-PD-L1 antibodies have been described in the art (see, e.g., U.S. Pat. Public. No. 20130122014 (Korman et al) and references cited therein); various anti-TIM-3 antibodies have been described in the art (see, e.g., U.S. Pat. Public. No. 20140044728 (Takayanagi et al) and references cited therein); and various anti-B7-H4 antibodies have been described in the art (see, e.g., U.S. Pat. Public. No. 20110085970 (Terrett et al) and references cited therein). Each of these references is hereby incorporated by reference in its entirety for the specific antibodies and sequences taught therein.

In various embodiments, IFN fusion partner can be an antibody, bispecific antibody, heterodimeric antibody, antibody fragment, a diabody, or protein or peptide that exhibit binding to an immune-checkpoint protein antigen that is present on the surface of an immune cell. In various embodiments, the immune-checkpoint protein antigen is selected from the group consisting of, but not limited to, CD279 (PD-1), CD274 (PDL-1), CD276, CD272, CD152, CD223 (LAG-3), CD40, SIRPα, CD47, OX-40, GITR, ICOS, CD27, 4-1BB, TIM-3, B7-H3, B7-H4, TIGIT, and VISTA.

### Linkers

In various embodiments, the fusion molecule is a recombinantly expressed fusion molecule. In various embodiments, the fusion molecules comprise an interferon molecule that is directly attached to the targeting moiety. In various embodiments, the fusion molecules comprise an IFN molecule that is attached to the targeting moiety via a peptide linker. In various embodiments, the linker may be an artificial sequence of between 5, 10, 15, 20, 30, 40 or more amino acids that are relatively free of secondary structure. In various embodiments, the linker is a rigid linker peptide of between 10, 15, 20, 30, 40 or more amino acids that display α-helical conformation and may act as rigid spacers between protein domains. In various embodiments, the peptide linker is a G/S rich linker. In various embodiments, the peptide linker is an alpha-helical linker. In various embodiments, the peptide linker is a glycine linker. In various embodiments, the peptide linker has the sequence set forth in SEQ ID NO: 20. In various embodiments, the peptide linker has the sequence set forth in SEQ ID NO: 21. In various embodiments, the peptide linker has the sequence set forth in SEQ ID NO: 22. In various embodiments, the peptide linker has the sequence set forth in SEQ ID NO: 23.

In various embodiments, the fusion molecules of the present disclosure will comprise the antibody and interferon molecule combinations recited in Table 4.

TABLE 4
Examples of TAA Ab-IFN Fusion Molecules

| Name | TAA Antibody | Linker | Interferon |
|---|---|---|---|
| FP-01 | Anti-PD-L1 (SEQ ID | None | Wt IFN-α2b (SEQ ID NO: 6) |
| | NOS: 15 and 16) | | |
| FP-02 | Anti-PD-L1 (SEQ ID | None | IFN-α2b (R149A) (SEQ ID NO: 7) |
| | NOS: 15 and 16) | | |
| FP-03 | Anti-PD-L1 (SEQ ID | None | Wt IFN-α5 (SEQ ID NO: 8) |
| | NOS: 15 and 16) | | |
| FP-04 | Anti-PD-L1 (SEQ ID | None | IFN-α5 (R150A) (SEQ ID NO: 9) |
| | NOS: 15 and 16) | | |
| FP-05 | Anti-PD-L1 (SEQ ID | None | IFN-α5 (A146D) (SEQ ID NO: 10) |
| | NOS: 15 and 16) | | |
| FP-06 | Anti-PD-L1 (SEQ ID | None | Wt con-IFN-α (SEQ ID NO: 1) |
| | NOS: 15 and 16) | | |
| FP-07 | Anti-PD-L1 (SEQ ID | None | Con-IFN-α (R150A) (SEQ ID NO: 2) |
| | NOS: 15 and 16) | | |
| FP-08 | Anti-PD-L1 (SEQ ID | None | Con-IFN-α (A146D) (SEQ ID NO: 3) |
| | NOS: 15 and 16) | | |
| FP-09 | Anti-PD-L1 (SEQ ID | None | Con-IFN-α (A146K) (SEQ ID NO: 4) |
| | NOS: 15 and 16) | | |
| FP-10 | Anti-PD-L1 (SEQ ID | None | Wt IFN-α6 (SEQ ID NO: 11) |
| | NOS: 15 and 16) | | |
| FP-11 | Anti-PD-L1 (SEQ ID | None | IFN-α6 (R150A) (SEQ ID NO: 12) |
| | NOS: 15 and 16) | | |
| FP-12 | Anti-PD-L1 (SEQ ID | None | IFN-α6 (A146D) (SEQ ID NO: 13) |
| | NOS: 15 and 16) | | |
| FP-13 | Anti-PD-L1 (SEQ ID | None | IFN-α6 (A146K) (SEQ ID NO: 14) |
| | NOS: 15 and 16) | | |
| FP-14 | Anti-PD-L1 (SEQ ID | G₈ | Con-IFN-α (R150A) (SEQ ID NO: 2) |
| | NOS: 15 and 17) | | |
| FP-15 | Anti-PD-L1 (SEQ ID | (G₄S)x3 | Con-IFN-α (R150A) (SEQ ID NO: 2) |
| | NOS: 15 and 17) | | |
| FP-16 | Anti-PD-L1 (SEQ ID | G₈ | Con-IFN-α (A146D) (SEQ ID NO: 3) |
| | NOS: 15 and 17) | | |
| FP-17 | Anti-PD-L1 (SEQ ID | (G₄S)x3 | Con-IFN-α (A146D) (SEQ ID NO: 3) |
| | NOS: 15 and 17) | | |
| FP-18 | Anti-PD-L1 (SEQ ID | G₈ | Con-IFN-α (A146D and R150A) (SEQ ID NO: 5) |
| | NOS: 15 and 17) | | |
| FP-19 | Anti-PD-L1 (SEQ ID | G₈ | Con-IFN-α (R150A) (SEQ ID NO: 2) |
| | NOS: 15, 18 and 19) | | |
| FP-20 | Anti-PD-L1 (SEQ ID | G₈ | Con-IFN-α (A146D and R150A) (SEQ ID NO: 5) |
| | NOS: 15, 18 and 19) | | |

### Polynucleotides

In another aspect, the present disclosure provides isolated nucleic acid molecules comprising a polynucleotide encoding IFN, an IFN variant, an IFN fusion protein, or an IFN variant fusion protein of the present disclosure. The subject nucleic acids may be single-stranded or double stranded. Such nucleic acids may be DNA or RNA molecules. DNA includes, for example, cDNA, genomic DNA, synthetic DNA, DNA amplified by PCR, and combinations thereof. Genomic DNA encoding IFN polypeptides is obtained from genomic libraries which are available for a number of species. Synthetic DNA is available from chemical synthesis of overlapping oligonucleotide fragments followed by assembly of the fragments to reconstitute part or all of the coding regions and flanking sequences. RNA may be obtained from prokaryotic expression vectors which direct high-level synthesis of mRNA, such as vectors using T7 promoters and RNA polymerase. cDNA is obtained from libraries prepared from mRNA isolated from various tissues that express IFN. The DNA molecules of the disclosure include full-length genes as well as polynucleotides and fragments thereof. The full-length gene may also include sequences encoding the N-terminal signal sequence. Such nucleic acids may be used, for example, in methods for making the novel IFN variants.

In various embodiments, the isolated nucleic acid molecules comprise the polynucleotides described herein, and further comprise a polynucleotide encoding at least one heterologous protein described herein. In various embodiments, the nucleic acid molecules further comprise polynucleotides encoding the linkers or hinge linkers described herein.

In various embodiments, the recombinant nucleic acids of the present disclosure may be operably linked to one or more regulatory nucleotide sequences in an expression construct. Regulatory sequences are art-recognized and are selected to direct expression of the IFN variant. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the present disclosure. The promoters may be either naturally occurring promoters, or hybrid promoters that combine elements of more than one promoter. An expression construct may be present in a cell on an episome, such as a plasmid, or the expression construct may be inserted in a chromosome. In various embodiments, the expression vector contains a selectable marker gene to allow the selection of transformed host cells. Selectable marker genes are well known in the art and will vary with the host cell used.

In another aspect of the present disclosure, the subject nucleic acid is provided in an expression vector comprising a nucleotide sequence encoding an IFN variant and operably linked to at least one regulatory sequence. The term “expression vector” refers to a plasmid, phage, virus or vector for expressing a polypeptide from a polynucleotide sequence. Vectors suitable for expression in host cells are readily available and the nucleic acid molecules are inserted into the vectors using standard recombinant DNA techniques. Such vectors can include a wide variety of expression control sequences that control the expression of a DNA sequence when operatively linked to it may be used in these vectors to express DNA sequences encoding an IFN variant. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., PhoS, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant IFN polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the B-gal containing pBlueBac III).

In various embodiments, a vector will be designed for production of the subject IFN variants in CHO cells, such as a Pcmv-Script vector (Stratagene, La Jolla, Calif.), pcDNA4 vectors (Invitrogen, Carlsbad, Calif.) and pCI-neo vectors (Promega, Madison, Wis.). As will be apparent, the subject gene constructs can be used to cause expression of the subject IFN variants in cells propagated in culture, e.g., to produce proteins, including fusion proteins or variant proteins, for purification.

This present disclosure also pertains to a host cell transfected with a recombinant gene including a nucleotide sequence coding an amino acid sequence for one or more of the subject IFN variant. The host cell may be any prokaryotic or eukaryotic cell. For example, an IFN variant of the present disclosure may be expressed in bacterial cells such as E. coli, insect cells (e.g., using a baculovirus expression system), yeast, or mammalian cells. Other suitable host cells are known to those skilled in the art.

Accordingly, the present disclosure further pertains to methods of producing the subject IFN variants. For example, a host cell transfected with an expression vector encoding an IFN variant can be cultured under appropriate conditions to allow expression of the IFN variant to occur. The IFN variant may be secreted and isolated from a mixture of cells and medium containing the IFN variant. Alternatively, the IFN variant may be retained cytoplasmically or in a membrane fraction and the cells harvested, lysed and the protein isolated. A cell culture includes host cells, media and other byproducts. Suitable media for cell culture is well known in the art.

The polypeptides and proteins of the present disclosure can be purified according to protein purification techniques are well known to those of skill in the art. These techniques involve, at one level, the crude fractionation of the proteinaceous and non-proteinaceous fractions. Having separated the peptide polypeptides from other proteins, the peptide or polypeptide of interest can be further purified using chromatographic and electrophoretic techniques to achieve partial or complete purification (or purification to homogeneity). The term “isolated polypeptide” or “purified polypeptide” as used herein, is intended to refer to a composition, isolatable from other components, wherein the polypeptide is purified to any degree relative to its naturally-obtainable state. A purified polypeptide therefore also refers to a polypeptide that is free from the environment in which it may naturally occur. Generally, “purified” will refer to a polypeptide composition that has been subjected to fractionation to remove various other components, and which composition substantially retains its expressed biological activity. Where the term “substantially purified” is used, this designation will refer to a peptide or polypeptide composition in which the polypeptide or peptide forms the major component of the composition, such as constituting about 50%, about 60%, about 70%, about 80%, about 85%, or about 90% or more of the proteins in the composition.

Various techniques suitable for use in purification will be well known to those of skill in the art. These include, for example, precipitation with ammonium sulphate, PEG, antibodies (immunoprecipitation) and the like or by heat denaturation, followed by centrifugation; chromatography such as affinity chromatography (Protein-A columns), ion exchange, gel filtration, reverse phase, hydroxylapatite, hydrophobic interaction chromatography; isoelectric focusing; gel electrophoresis; and combinations of these techniques. As is generally known in the art, it is believed that the order of conducting the various purification steps may be changed, or that certain steps may be omitted, and still result in a suitable method for the preparation of a substantially purified polypeptide.

### Pharmaceutical Compositions

In another aspect, the present disclosure provides a pharmaceutical composition comprising the con-IFN-α mutants, or bifunctional con-IFN-α mutant fusion proteins, in admixture with a pharmaceutically acceptable carrier. Such pharmaceutically acceptable carriers are well known and understood by those of ordinary skill and have been extensively described (see, e.g., Remington's Pharmaceutical Sciences, 18th Edition, A. R. Gennaro, ed., Mack Publishing Company, 1990). The pharmaceutically acceptable carriers may be included for purposes of modifying, maintaining or preserving, for example, the pH, osmolarity, viscosity, clarity, color, isotonicity, odor, sterility, stability, rate of dissolution or release, adsorption or penetration of the composition. Such pharmaceutical compositions may influence the physical state, stability, rate of in vivo release, and rate of in vivo clearance of the polypeptide. Suitable pharmaceutically acceptable carriers include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute thereof. In one embodiment of the present disclosure, compositions may be prepared for storage by mixing the selected composition having the desired degree of purity with optional formulation agents (Remington's Pharmaceutical Sciences, supra) in the form of a lyophilized cake or an aqueous solution. Further, the therapeutic composition may be formulated as a lyophilizate using appropriate excipients such as sucrose. The optimal pharmaceutical composition will be determined by one of ordinary skill in the art depending upon, for example, the intended route of administration, delivery format, and desired dosage.

When parenteral administration is contemplated, the therapeutic pharmaceutical compositions may be in the form of a pyrogen-free, parenterally acceptable aqueous solution comprising the desired con-IFN-α mutants, or bifunctional con-IFN-α mutant fusion proteins, in a pharmaceutically acceptable vehicle. A particularly suitable vehicle for parenteral injection is sterile distilled water in which a polypeptide is formulated as a sterile, isotonic solution, properly preserved. In various embodiments, pharmaceutical formulations suitable for injectable administration may be formulated in aqueous solutions, preferably in physiologically compatible buffers such as Hanks' solution, Ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Optionally, the suspension may also contain suitable stabilizers or agents to increase the solubility of the compounds and allow for the preparation of highly concentrated solutions.

In various embodiments, the therapeutic pharmaceutical compositions may be formulated for targeted delivery using a colloidal dispersion system. Colloidal dispersion systems include macromolecule complexes, nanocapsules, microspheres, beads, and lipid-based systems including oil-in-water emulsions, micelles, mixed micelles, and liposomes. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

In various embodiments, oral administration of the pharmaceutical compositions is contemplated. Pharmaceutical compositions that are administered in this fashion can be formulated with or without those carriers customarily used in the compounding of solid dosage forms such as tablets and capsules. In solid dosage forms for oral administration (capsules, tablets, pills, dragees, powders, granules, and the like), one or more therapeutic compounds of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof Besides inert diluents, the oral compositions can also include adjuvants such as wetting agents, emulsifying and suspending agents, sweetening, flavoring, coloring, perfuming, and preservative agents.

In various embodiments, topical administration of the pharmaceutical compositions, either to skin or to mucosal membranes, is contemplated. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur. Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required. The ointments, pastes, creams and gels may contain, in addition to a subject compound of the disclosure (e.g., a con-IFN-α mutant), excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Additional pharmaceutical compositions contemplated for use herein include formulations involving polypeptides in sustained- or controlled-delivery formulations. Techniques for formulating a variety of other sustained- or controlled-delivery means, such as liposome carriers, bio-erodible microparticles or porous beads and depot injections, are also known to those skilled in the art.

An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the polypeptide is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.001 mg/kg to up to about 100 mg/kg or more, depending on the factors mentioned above. Polypeptide compositions may be preferably injected or administered intravenously. Long-acting pharmaceutical compositions may be administered every three to four days, every week, or biweekly depending on the half-life and clearance rate of the particular formulation. The frequency of dosing will depend upon the pharmacokinetic parameters of the polypeptide in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

The route of administration of the pharmaceutical composition is in accord with known methods, e.g. orally, through injection by intravenous, intraperitoneal, intracerebral (intra-parenchymal), intracerebroventricular, intramuscular, intra-ocular, intraarterial, intraportal, intralesional routes, intramedullary, intrathecal, intraventricular, transdermal, subcutaneous, or intraperitoneal or intratumorally; as well as intranasal, enteral, topical, sublingual, urethral, vaginal, or rectal means, by sustained release systems or by implantation devices. Where desired, the compositions may be administered by bolus injection or continuously by infusion, or by implantation device. Alternatively, or additionally, the composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed-release bolus, or continuous administration.

### Therapeutic Uses

In another aspect, the present disclosure provides for a method of treating a type I interferon mediated disorder selected from the group consisting of: cancer, infectious diseases, immune disorders, inflammatory diseases or conditions, and autoimmune diseases.

As used herein, cancer refers to any uncontrolled growth of cells that may interfere with the normal functioning of the bodily organs and systems and includes both primary and metastatic tumors. Primary tumors or cancers that migrate from their original location and seed vital organs can eventually lead to the death of the subject through the functional deterioration of the affected organs. A metastasis is a cancer cell or group of cancer cells, distinct from the primary tumor location, resulting from the dissemination of cancer cells from the primary tumor to other parts of the body. Metastases may eventually result in death of a subject. For example, cancers can include benign and malignant cancers, polyps, hyperplasia, as well as dormant tumors or micrometastases.

In another aspect, the present disclosure provides for a method of treating cancer cells in a subject, comprising administering to said subject a therapeutically effective amount (either as monotherapy or in a combination therapy regimen) of a con-IFN-α mutant, or bifunctional con-IFN-α mutant fusion protein of the present disclosure in pharmaceutically acceptable carrier, wherein such administration inhibits the growth and/or proliferation of a cancer cell. Specifically, a con-IFN-α mutant, or bifunctional con-IFN-α mutant fusion protein of the present disclosure is useful in treating disorders characterized as cancer. Such disorders include, but are not limited to solid tumors, such as cancers of the breast, respiratory tract, brain, reproductive organs, digestive tract, urinary tract, eye, liver, skin, head and neck, thyroid, parathyroid and their distant metastases, lymphomas, sarcomas, multiple myeloma and leukemia. Examples of breast cancer include, but are not limited to invasive ductal carcinoma, invasive lobular carcinoma, ductal carcinoma in situ, and lobular carcinoma in situ. Examples of cancers of the respiratory tract include, but are not limited to, small-cell and non-small-cell lung carcinoma, as well as bronchial adenoma and pleuropulmonary blastoma. Examples of brain cancers include, but are not limited to, brain stem and hypothalmic glioma, cerebellar and cerebral astrocytoma, medulloblastoma, ependymoma, as well as neuroectodermal and pineal tumor. Tumors of the male reproductive organs include, but are not limited to, prostate and testicular cancer. Tumors of the female reproductive organs include, but are not limited to endometrial, cervical, ovarian, vaginal, and vulvar cancer, as well as sarcoma of the uterus. Tumors of the digestive tract include, but are not limited to anal, colon, colorectal, esophageal, gallbladder, gastric, pancreatic, rectal, small-intestine, and salivary gland cancers. Tumors of the urinary tract include, but are not limited to, bladder, penile, kidney, renal pelvis, ureter, and urethral cancers. Eye cancers include, but are not limited to, intraocular melanoma and retinoblastoma. Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma. Skin cancers include, but are not limited to squamous cell carcinoma, Kaposi's sarcoma, malignant melanoma, Merkel cell skin cancer, and non-melanoma skin cancer. Head-and-neck cancers include, but are not limited to nasopharyngeal cancer, and lip and oral cavity cancer. Lymphomas include, but are not limited to AIDS-related lymphoma, non-Hodgkin's lymphoma, cutaneous T-cell lymphoma, Hodgkin's disease, and lymphoma of the central nervous system. Sarcomas include, but are not limited to, sarcoma of the soft tissue, osteosarcoma, malignant fibrous histiocytoma, lymphosarcoma, and rhabdomyosarcoma. Leukemias include, but are not limited to acute myeloid leukemia, acute lymphoblastic leukemia, various lymphocytic leukemia, various myelogenous leukemia, and hairy cell leukemia. In various embodiments, the cancer will be a cancer with high expression of TGF-β family member, such as activin A, myostatin, TGF-β and GDF15, e.g., pancreatic cancer, gastric cancer, ovarian cancer, colorectal cancer, melanoma leukemia, lung cancer, prostate cancer, brain cancer, bladder cancer, and head-neck cancer.

In various embodiments, the present con-IFN-α mutants, or bifunctional con-IFN-α mutant fusion proteins can be utilized to promote growth inhibition and/or proliferation of a cancerous tumor cell. These methods may inhibit or prevent the growth of the cancer cells of said subject, such as for example, by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. As a result, where the cancer is a solid tumor, the modulation may reduce the size of the solid tumor by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

The inhibition of the cancer cell proliferation can be measured by cell-based assays, such as bromodeoxyuridine (BRDU) incorporation (Hoshino et al., Int. J. Cancer 38, 369, 1986; Campana et al., J. Immunol. Meth. 107:79, 1988; [³H]-thymidine incorporation (Chen, J., Oncogene 13:1395-403, 1996; Jeoung, J., J. Biol. Chem. 270:18367-73, 1995; the dye Alamar Blue (available from Biosource International) (Voytik-Harbin et al., In Vitro Cell Dev Biol Anim 34:239-46, 1998). The anchorage independent growth of cancer cells is assessed by colony formation assay in soft agar, such as by counting the number of cancer cell colonies formed on top of the soft agar (see Examples and Sambrook et al., Molecular Cloning, Cold Spring Harbor, 1989).

The inhibition of cancer cell growth in a subject may be assessed by monitoring the cancer growth in a subject, for example in an animal model or in human subjects. One exemplary monitoring method is tumorigenicity assays. In one example, a xenograft comprises human cells from a pre-existing tumor or from a tumor cell line. Tumor xenograft assays are known in the art and described herein (see, e.g., Ogawa et al., Oncogene 19:6043-6052, 2000). In another embodiment, tumorigenicity is monitored using the hollow fiber assay, which is described in U.S. Pat. No. 5,698,413, which is incorporated herein by reference in its entirety.

The percentage of the inhibition is calculated by comparing the cancer cell proliferation, anchorage independent growth, or cancer cell growth under modulator treatment with that under negative control condition (typically without modulator treatment). For example, where the number of cancer cells or cancer cell colonies (colony formation assay), or PRDU or [³H]-thymidine incorporation is A (under the treatment of modulators) and C (under negative control condition), the percentage of inhibition would be (C−A)/C×100%.

Examples of tumor cell lines derived from human tumors and available for use in the in vitro and in vivo studies include, but are not limited to, B lymphoblast cell lines (e.g., Daudi cell lines); leukemia cell lines (e.g., CCRF-CEM, HL-60(TB), K-562, MOLT-4, RPM1-8226, SR, P388 and P388/ADR); non-small cell lung cancer cell lines (e.g., A549/ATCC, EKVX, HOP-62, HOP-92, NCI-H226, NCI-H23, NCI-H322M, NCI-H460, NCI-H522 and LXFL 529); small cell lung cancer cell lines (e.g., DMS 114 and SHP-77); colon cancer cell lines (e.g., COLO 205, HCC-2998, HCT-116, HCT-15, HT29, KM12, SW-620, DLD-1 and KM20L2); central nervous system (CNS) cancer cell lines (e.g., SF-268, SF-295, SF-539, SNB-19, SNB-75, U251, SNB-78 and XF 498); melanoma cell lines (e.g., LOX I MVI, MALME-3M, M14, SK-MEL-2, SK-MEL-28, SK-MEL-5, UACC-257, UACC-62, RPMI-7951 and M19-MEL); ovarian cancer cell lines (e.g., IGROV1, OVCAR-3, OVCAR-4, OVCAR-5, OVCAR-8 and SK-OV-3); renal cancer cell lines (e.g., 786-0, A498, ACHN, CAKI-1, RXF 393, SN12C, TK-10, UO-31, RXF-631 and SN12K1); prostate cancer cell lines (e.g., PC-3 and DU-145); breast cancer cell lines (e.g., MCF7, NCI/ADR-RES, MDA-MB-231/ATCC, HS 578T, MDA-MB-435, BT-549, T-47D and MDA-MB-468); and thyroid cancer cell lines (e.g., SK-N-SH).

Chronic hepatitis B virus (HBV) infection is a major cause of hepatocellular carcinoma (HCC) worldwide and its most important cause in Asia. Seventy-five percent of all chronic HBV infections occur in Asia and the prevalence in Taiwan is 15-20%, where more than 90% of the adult population has been infected with HBV in the past (Chen et al., J. Formos. Med. Assoc. (2007) 106 (2):148-55). Current clinically approved treatment options are nucleos(t)ide analogs and interferon-α. Nucleos(t)ide analogs control but do not cure hepatitis B and require expensive long-term treatment potentially associated with the emergence of resistant viruses. Interferon-α therapy is limited by side-effects and is only curative in 15-20% of patients. The virus is divided into four major serotypes (adr, adw, ayr, ayw) that induce differential antibody responses based on antigenic epitopes present on its envelope proteins, and into (at least) eight genotypes (A-I) according to overall nucleotide sequence variation of the genome. The genotypes have a distinct geographical distribution and are used in tracing the evolution and transmission of the virus. Differences between genotypes affect the disease severity, course and likelihood of complications, and response to treatment and possibly vaccination. Furthermore, subgenotypes, e.g. A1-5, exist. In Central Europe and the United States, the predominant genotype is A2. However, only 1% of the infected humans carry the A2 subgenotype, while the majority of patients carry HBV of the genotype B, C, or D. It has been shown that conventional HBV vaccines provide a better protection against HBV of the same (sub) genotype as the HBV antigens comprised in the vaccine than to other (sub) genotypes.

In another aspect, the present disclosure provides for a method of treating HBV infection in a subject, comprising administering to said subject a therapeutically effective amount (either as monotherapy or in a combination therapy regimen) of a con-IFN-α mutant, or bifunctional con-IFN-α mutant fusion protein of the present disclosure in pharmaceutically acceptable carrier, wherein such administration protects the cell from virus infection.

In various embodiments, the present con-IFN-α mutants, or bifunctional con-IFN-α mutant fusion proteins can be utilized to protect cells from a viral challenge. These methods may inhibit or prevent the viral infection of said subject, such as for example, by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

The protection from viral challenge can be measured by treating cells with drug and then challenging cells with HBV and monitoring infection rates. The rate of infection can be assessed by measuring expression of hepatitis B surface antigen (HBsAg) and HBV DNA.

Examples of cells that can be used to monitor infection rates after drug treatment and viral challenge include, but are not limited to, human fetal hepatocytes, human adult hepatocytes, primary tree shrew hepatocytes, HepaRG cells, induced pluripotent stem cell-derived human hepatocytes, and NTCP-overexpressing hepatoma cell lines (see, e.g., Xu et al., Virology Journal 18(1):105, 2021).

In various embodiments, the present con-IFN-α mutants, or bifunctional con-IFN-α mutant fusion proteins can be utilized to treat HBV-infected cells. These methods may treat the viral infection of said subject, such as for example, by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%.

The efficacy of antiviral treatment of HBV infected cells can be measured by treating HBV infected cells with drug and then measuring markers of HBV infection. Infection can be assessed by measuring expression HBsAg and HBV DNA.

Examples of cells that can be used to monitor antiviral treatment of HBV infected cells include, but are not limited to, HepG2.2.15 cells, HepAD38 cells, HepDE19 cells and HepDES19 cells (see, e.g., Xu et al., Virology Journal 18(1):105, 2021).

Hepatitis C is a blood-borne, infectious, viral disease that is caused by an RNA virus belonging to the Hepacivirus genus in the Flaviviridae family called HCV. The enveloped HCV virion contains a positive stranded RNA genome encoding all known virus-specific proteins in a single, uninterrupted, open reading frame. The open reading frame comprises approximately 9500 nucleotides and encodes a single large polyprotein of about 3000 amino acids. The polyprotein comprises a core protein, envelope proteins E1 and E2, a membrane bound protein p7, and the non-structural proteins NS2, NS3, NS4A, NS4B, NS5A and NS5B. At least six different HCV genotypes (with several subtypes within each genotype) are known to date. In North America, HCV genotype 1a predominates, followed by HCV genotypes 1b, 2a, 2b, and 3a. In the United States, HCV genotypes 1, 2, and 3 are the most common, with about 80% of the hepatitis C patients having HCV genotype 1. In Europe, HCV genotype 1b is predominant, followed by HCV genotypes 2a, 2b, 2c, and 3a. HCV genotypes 4 and 5 are found almost exclusively in Africa. The patient's HCV genotype is clinically important in determining the patient's potential response to therapy and the required duration of such therapy.

Chronic HCV infection is associated with progressive liver pathology, including cirrhosis and hepatocellular carcinoma. In the past, chronic hepatitis C was treated with peginterferon-alpha in combination with ribavirin, which had substantial limitations of efficacy and tolerability. Recently, Glecaprevir-pibrentasvir was approved for treatment of chronic HCV infection in treatment-naive individuals without cirrhosis for a duration lasting only eight weeks. Further, all other currently approved treatments, sofosbuvir, ledipasvir, velpatasvir, voxilaprevir, daclatasvir, elbasvir, grazoprevir, simeprevir are indicated for the treatment of chronic HCV. While numerous drugs are indicated for treatment of chronic HCV, no drug thus far is indicated for acute treatment of HCV. Moreover, since the goal of World Health Organization is to eliminate HCV as a public threat by 2030, therefore, there is a need for new therapies and treatment options to treat acute HCV infection, wherein duration is further shortened for compliance, without negative impact on efficacy, viral breakthrough, resistance, recurrent viraemia, virological relapse or reinfection.

There are two treatment regimens that are primarily used to treat hepatitis C: monotherapy (using an interferon agent—either a “conventional” or longer-acting pegylated interferon) and combination therapy (using an interferon agent and ribavirin). Interferon, which is injected into the bloodstream, works by bolstering the immune response to HCV; and ribavirin, which is taken orally, is believed to work by preventing HCV replication. Taken alone, ribavirin does not effectively suppress HCV levels, but an interferon/ribavirin combination is more effective than interferon alone. Typically, hepatitis C is treated with a combination of pegylated interferon alpha and ribavirin for a period of 24 or 48 weeks, depending on the HCV genotype. Acute HCV infection may also be defined as within six month of ALT>10×ULN. Methods of determining ALT and ULN are known in the art.

In another aspect, the present disclosure provides for a method of treating HCV infection in a subject, comprising administering to said subject a therapeutically effective amount (either as monotherapy or in a combination therapy regimen) of a con-IFN-α mutant, or bifunctional con-IFN-α mutant fusion protein of the present disclosure in pharmaceutically acceptable carrier, wherein such administration protects the cell from virus infection.

“Therapeutically effective amount” or “therapeutically effective dose” refers to that amount of the therapeutic agent being administered which will relieve to some extent one or more of the symptoms of the disorder being treated. A therapeutically effective dose can be estimated initially from cell culture assays by determining an EC₅₀. A dose can then be formulated in animal models to achieve a circulating plasma concentration range that includes the EC₅₀ as determined in cell culture. Such information can be used to more accurately determine useful doses in humans. Levels in plasma may be measured, for example, by HPLC. The exact composition, route of administration and dosage can be chosen by the individual physician in view of the subject's condition.

Dosage regimens can be adjusted to provide the optimum desired response (e.g., a therapeutic or prophylactic response). For example, a single bolus can be administered, several divided doses (multiple or repeat or maintenance) can be administered over time and the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present disclosure will be dictated primarily by the unique characteristics of the antibody and the particular therapeutic or prophylactic effect to be achieved.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present disclosure.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this disclosure may be based on a variety of factors, including the type of disease, the age, weight, sex, medical condition of the subject, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-subject dose-escalation as determined by the skilled artisan. Determining appropriate dosages and regimens are well-known in the relevant art and would be understood to be encompassed by the skilled artisan once provided the teachings disclosed herein.

An exemplary, non-limiting daily dosing range for a therapeutically or prophylactically effective amount of a con-IFN-α mutant, or bifunctional con-IFN-α mutant fusion protein of the disclosure can be 0.001 to 100 mg/kg, 0.001 to 90 mg/kg, 0.001 to 80 mg/kg, 0.001 to 70 mg/kg, 0.001 to 60 mg/kg, 0.001 to 50 mg/kg, 0.001 to 40 mg/kg, 0.001 to 30 mg/kg, 0.001 to 20 mg/kg, 0.001 to 10 mg/kg, 0.001 to 5 mg/kg, 0.001 to 4 mg/kg, 0.001 to 3 mg/kg, 0.001 to 2 mg/kg, 0.001 to 1 mg/kg, 0.010 to 50 mg/kg, 0.010 to 40 mg/kg, 0.010 to 30 mg/kg, 0.010 to 20 mg/kg, 0.010 to 10 mg/kg, 0.010 to 5 mg/kg, 0.010 to 4 mg/kg, 0.010 to 3 mg/kg, 0.010 to 2 mg/kg, 0.010 to 1 mg/kg, 0.1 to 50 mg/kg, 0.1 to 40 mg/kg, 0.1 to 30 mg/kg, 0.1 to 20 mg/kg, 0.1 to 10 mg/kg, 0.1 to 5 mg/kg, 0.1 to 4 mg/kg, 0.1 to 3 mg/kg, 0.1 to 2 mg/kg, 0.1 to 1 mg/kg, 1 to 50 mg/kg, 1 to 40 mg/kg, 1 to 30 mg/kg, 1 to 20 mg/kg, 1 to 10 mg/kg, 1 to 5 mg/kg, 1 to 4 mg/kg, 1 to 3 mg/kg, 1 to 2 mg/kg, or 1 to 1 mg/kg body weight. It is to be noted that dosage values may vary with the type and severity of the conditions to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Toxicity and therapeutic index of the pharmaceutical compositions of the disclosure can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effective dose is the therapeutic index and it can be expressed as the ratio LD₅₀/ED₅₀. Compositions that exhibit large therapeutic indices are generally preferred.

The dosing frequency of the administration of the con-IFN-α mutant, or bifunctional con-IFN-α mutant fusion protein pharmaceutical composition depends on the nature of the therapy and the particular disease being treated. The subject can be treated at regular intervals, such as twice weekly, weekly or monthly, until a desired therapeutic result is achieved. Exemplary dosing frequencies include but are not limited to: once weekly without break; once every 2 weeks; once every 3 weeks; weekly without break for 2 weeks, then monthly; weekly without break for 3 weeks, then monthly; monthly; once every other month; once every three months; once every four months; once every five months; or once every six months, or yearly.

### Combination Therapy

In another aspect, the present disclosure provides a method for treating cancer or cancer metastasis in a subject, comprising administering a therapeutically effective amount of the pharmaceutical compositions of the invention in combination with a second therapy, including, but not limited to immunotherapy, cytotoxic chemotherapy, small molecule kinase inhibitor targeted therapy, surgery, radiation therapy, and stem cell transplantation. For example, such methods can be used in prophylactic cancer prevention, prevention of cancer recurrence and metastases after surgery, and as an adjuvant of other conventional cancer therapy. The present disclosure recognizes that the effectiveness of conventional cancer therapies (e.g., chemotherapy, radiation therapy, phototherapy, immunotherapy, and surgery) can be enhanced through the use of the combination methods described herein.

A wide array of conventional compounds has been shown to have anti-neoplastic activities. These compounds have been used as pharmaceutical agents in chemotherapy to shrink solid tumors, prevent metastases and further growth, or decrease the number of malignant T-cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

In various embodiments, a second anti-cancer agent, such as a chemotherapeutic agent, will be administered to the patient. The list of exemplary chemotherapeutic agent includes, but is not limited to, daunorubicin, dactinomycin, doxorubicin, bleomycin, mitomycin, nitrogen mustard, chlorambucil, melphalan, cyclophosphamide, 6-mercaptopurine, 6-thioguanine, bendamustine, cytarabine (CA), 5-fluorouracil (5-FU), floxuridine (5-FUdR), methotrexate (MTX), colchicine, vincristine, vinblastine, etoposide, teniposide, cisplatin, carboplatin, oxaliplatin, pentostatin, cladribine, cytarabine, gemcitabine, pralatrexate, mitoxantrone, diethylstilbestrol (DES), fluradabine, ifosfamide, hydroxyureataxanes (such as paclitaxel and doxetaxel) and/or anthracycline antibiotics, as well as combinations of agents such as, but not limited to, DA-EPOCH, CHOP, CVP or FOLFOX. In various embodiments, the dosages of such chemotherapeutic agents include, but is not limited to, about any of 10 mg/m², 20 mg/m², 30 mg/m², 40 mg/m², 50 mg/m², 60 mg/m², 75 mg/m², 80 mg/m², 90 mg/m², 100 mg/m², 120 mg/m², 150 mg/m², 175 mg/m², 200 mg/m², 210 mg/m², 220 mg/m², 230 mg/m², 240 mg/m², 250 mg/m², 260 mg/m², and 300 mg/m².

In various embodiments, the combination therapy methods of the present disclosure may further comprise administering to the subject a therapeutically effective amount of immunotherapy, including, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, OX-40, CD137, GITR, LAG3, TIM-3, SIRP, CD47, CD40 Siglec 8, Siglec 9, Siglec 15, TIGIT and VISTA; treatment using bispecific T cell engaging antibodies (BiTE®) such as blinatumomab: treatment involving administration of biological response modifiers such as IL-2, IL-12, IL-15, IL-21, GM-CSF, IFN-α, IFN-β and IFN-γ; treatment using therapeutic vaccines such as sipuleucel-T; treatment using Bacilli Calmette-Guerin (BCG); treatment using dendritic cell vaccines, or tumor antigen peptide vaccines; treatment using T-cells, chimeric antigen receptor (CAR)-T cells, or iPS-induced T-cells or iPS-induced CAR-T cells; treatment using NK cells, CAR-NK cells or iPS-induced NK cells, or iPS-induced CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod; wherein the combination therapy provides increased killing of tumor cells, i.e., a synergy exists between con-IFN-α variants and the immunotherapy when co-administered.

Immunotherapy focused on utilization of depleting antibodies to specific tumor antigens have been explored with much success (see, e.g., reviews by Blattman and Greenberg, Science, 305:200, 2004; Adams and Weiner, Nat Biotech, 23:1147, 2005). A few examples of such tumor antigen-specific, depleting antibodies are HERCEPTIN® (anti-Her2/neu mAb) (Baselga et al., J Clin Oncology, Vol 14:737, 1996; Baselga et al., Cancer Research, 58:2825, 1998; Shak, Semin. Oncology, 26 (Suppl12):71, 1999; Vogal et al. J Clin Oncology, 20:719, 2002); and RITUXAN® (anti-CD20 mAb) (Colombat et al., Blood, 97:101, 2001). Unfortunately, while clearly having made a mark in oncology treatment, as monotherapy they generally work in only about 30% of the individuals and with a partial response. Moreover, many individuals eventually become refractory or relapse after treatment with these antibody-containing regimens.

Treatment using agonistic, antagonistic, or blocking antibodies to co-stimulatory or co-inhibitory molecules (immune checkpoints) has been an area of extensive research and clinical evaluation. Under normal physiological conditions, immune checkpoints are crucial for the maintenance of self-tolerance (that is, the prevention of autoimmunity) and protect tissues from damage when the immune system is responding to pathogenic infection. It is now also clear that tumors co-opt certain immune-checkpoint pathways as a major mechanism of immune resistance, particularly against T cells that are specific for tumor antigens (Pardoll DM., Nat Rev Cancer, 12:252-64, 2012). Accordingly, treatment utilizing antibodies to immune checkpoint molecules including, e.g., CTLA-4 (ipilimumab), PD-1 (nivolumab; pembrolizumab; cemiplimab) and PD-L1 (atezolizumab; durvalumab; avelumab) (see, e.g, Philips and Atkins, International Immunology, 27(1); 39-46, October 2014), and OX-40, CD137, GITR, LAG3, TIGIT, TIM-3, and VISTA (see, e.g., Sharon et al., Chin J Cancer., 33(9): 434-444, September 2014; Hodi et al., N Engl J Med, 2010; Topalian et al., N Engl J Med, 366:2443-54) are being evaluated or have been approved by the FDA as new, alternative immunotherapies to treat patients with proliferative diseases such as cancer, and in particular, patients with refractory and/or recurrent cancers.

Treatment using chimeric antigen receptor (CAR) T cell therapy is an immunotherapy in which the patient's own T cells are isolated in the laboratory, redirected with a synthetic receptor to recognize a particular antigen or protein, and reinfused into the patient. CARs are synthetic molecules that minimally contain: (1) an antigen-binding region, typically derived from an antibody, (2) a transmembrane domain to anchor the CAR into the T cells, and (3) 1 or more intracellular T cell signaling domains. A CAR redirects T cell specificity to an antigen in a human leukocyte antigen (HLA)-independent fashion, and overcomes issues related to T cell tolerance (Kalos M and June C H, Immunity, 39(1):49-60, 2013). A new wave of excitement surrounding CAR-T cell therapy began in August 2011, when investigators from the University of Pennsylvania (Penn) published a report on 3 patients with refractory chronic lymphocytic leukemia (CLL) who had long-lasting remissions after a single dose of CAR T cells directed to CD19 (Porter D L, et al., N Engl J Med., 365(8):725-733, 2011). After successful clinical trials in ALL, NHL, and multiple myeloma five CAR-T therapies have been approved by the FDA. These include Abecma, Breyanzi, Kymriah, Tecartus, and Yescarta and all except for Abecma, which targets the B-cell maturation antigen (BCMA), target the CD19 antigen expressed on malignant B cells. Next generation CAR-T cell therapies are being developed and tested in the clinic by many pharmaceutical and biotechnology companies and are designed to improve upon the toxicity, efficacy, and manufacturability of the first-generation cellular therapies (see, e.g., Sterner and Sterner, Blood Cancer Journal 11:69, 2021; Larson and Maus, Nature Reviews Cancer 21:145-161, 2021)

In contrast to donor T cells, natural killer (NK) cells are known to mediate anti-cancer effects without the risk of inducing graft-versus-host disease (GvHD). Accordingly, alloreactive NK cells are now also the focus of considerable interest as suitable and powerful effector cells for cellular therapy of cancer. Several human NK cell lines have been established, e.g., NK-92, HANK-1, KHYG-1, NK-YS, NKG, YT, YTS, NKL and NK3.3 (Kornbluth, J., et al., J. Immunol. 134, 728-735, 1985; Cheng, M. et al., Front. Med. 6:56, 2012) and various CAR expressing NK cells (CAR-NK) have been generated. Immunotherapy using CAR expressing NK cells (CAR-NK) is an active area of research and clinical evaluation (see, e.g., Glienke et al., Front Pharmacol, 6(21):1-7, February 2015).

Bispecific T-cell engager molecules (BiTE®s) constitute a class of bispecific single-chain antibodies for the polyclonal activation and redirection of cytotoxic T cells against pathogenic target cells. BiTE®s are bispecific for a surface target antigen on cancer cells, and for CD3 on T cells. BiTE®s are capable of connecting any kind of cytotoxic T cell to a cancer cell, independently of T-cell receptor specificity, costimulation, or peptide antigen presentation. a unique set of properties that have not yet been reported for any other kind of bispecific antibody construct, namely extraordinary potency and efficacy against target cells at low T-cell numbers without the need for T-cell co-stimulation (Baeuerle et al., Cancer Res, 69(12):4941-4, 2009). BiTE antibodies have so far been constructed to many different target antigens, including CD19, EpCAM, Her2/neu, EGFR, CD66e (or CEA, CEACAM5), CD33, EphA2, MCSP (or HMW-MAA) (Id.), BCMA, CLDN18.2, DLL3, FLT3, MUC17, and PSMA. Currently, the only FDA approved BiTE® antibody is Blincyto (blinatumomab) but many are being tested in early clinical trials.

In another aspect, the present disclosure provides a method for treating an infectious disease in a subject, comprising administering a therapeutically effective amount of the pharmaceutical compositions of the invention in combination with a second agent/therapy selected from the group consisting of Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuvirtide, Etravirine, Famciclovir, and Foscarnet. In some embodiments, the anti-infective is an anti-bacterial agent including, but not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); and carbapenem antibiotics (ertapenem, doripenem, imipenem/cilastatin, and meropenem). In some embodiments, the anti-infectives include anti-malarial agents (e.g., chloroquine, quinine, mefloquine, primaquine, doxycycline, artemether/lumefantrine, atovaquone/proguanil and sulfadoxine/pyrimethamine), metronidazole, tinidazole, ivermectin, pyrantel pamoate, and albendazole.

In another aspect, the present disclosure provides a method for treating HCV in a subject, comprising administering a therapeutically effective amount of the pharmaceutical compositions of the invention in combination with a second therapy selected from the group consisting of protease inhibitors, polymerase inhibitors, direct-acting antivirals, ribavirin and pegylated interferon. In various embodiments, the second therapy is selected from the group consisting of: Mavyet (glecaprevir/pibrentasvir), Epclusa (sofosbuvir/velpatasvir), Vosevi (sofosbuvir/velpatasvir/voxilapresvir), Harvoni (ledipasvir/sofosbuvir), Sovaldi (sofosbuvir), and Zepatier (elbasvir/grazoprevir). Exemplary formulations of ribavirin include COPEGUS®, REBETOL®, and RIBASPHERE®. An exemplary pro-drug of ribavirin is taribavirin having the chemical name of 1-β-D-ribofuranosyl-1,2,4-triazole-3-carboxamidine. Ribavirin and taribavirin may be administered in accordance with ribavirin and taribavirin administration well known in the art. For example, ribavirin or taribavirin may be administered in a total daily dose of from about 5 mg to about 1500 mg.

In another aspect, the present disclosure provides a method for treating HBV in a subject, comprising administering a therapeutically effective amount of the pharmaceutical compositions of the invention in combination with a second therapy selected from the group consisting of: treatment using nucleo(t)side analogs such as Tenofovir disoproxil fumarate (TDF), Tenofovir alafenamide (TAF), Lamivudine, Adefovir dipivoxil, Entecavir (ETV), Telbivudine, AGX-1009, emtricitabine, clevudine, ritonavir, dipivoxil, lobucavir, famvir, FTC, N-Acetyl-Cysteine (NAC), PC1323, theradigm-HBV, thymosin-alpha, and ganciclovir, besifovir (ANA-380/LB-80380), and tenofovir-exaliades (TLX/CMX157); treatment using other antiviral drugs such as siRNA, antisense oligonucleotides, capsid assembly modulators, and polymerase inhibitors; treatment using immunomodulatory therapies such as TLR7 agonist, TLR8 agonist, STING agonist, RIG-I activator, PD-1 blocking antibody, PD-L1 blocking antibody, TIM-3 blocking antibody, LAG-3 blocking antibody, and CTLA-4 blocking antibody; treatment using therapeutic vaccines against HBV antigens; and treatment using adoptive cellular therapy such as HBV-specific CAR-T cells, HBV-specific TCR-T cells, and other HBV-specific cellular therapies.

In various embodiments, the combination therapy comprises administering a con-IFN-α mutant, or bifunctional con-IFN-α mutant fusion protein composition and the second therapy or second agent composition simultaneously, either in the same pharmaceutical composition or in separate pharmaceutical composition. In various embodiments, a con-IFN-α mutant, or bifunctional con-IFN-α mutant fusion protein composition and the second therapy or second agent composition are administered sequentially, i.e., a con-IFN-α mutant, or bifunctional con-IFN-α mutant fusion protein composition is administered either prior to or after the administration of the second therapy or second agent composition. In various embodiments, the administrations of a con-IFN-α mutant, or bifunctional con-IFN-α mutant fusion protein and the second therapy or second agent composition are concurrent, i.e., the administration period of a con-IFN-α mutant, or bifunctional con-IFN-α mutant fusion protein composition and the second therapy or second agent composition overlap with each other. In various embodiments, the administrations of a con-IFN-α mutant, or bifunctional con-IFN-α mutant fusion protein composition and the second therapy or second agent composition are non-concurrent. For example, in various embodiments, the administration of a con-IFN-α mutant, or bifunctional con-IFN-α mutant fusion protein composition is terminated before the second therapy or second agent composition is administered. In various embodiments, the administration of the second therapy or second agent composition is terminated before a con-IFN-α mutant, or bifunctional con-IFN-α mutant fusion protein composition is administered.

The following examples are offered to more fully illustrate the disclosure but are not construed as limiting the scope thereof.

### Example 1

### Construction and Production of PD-L1 Ab-IFN Mutant Fusion Constructs

Sequences for IFN-α2b (Accession #P01563), IFN-α5 (Accession #P01569, and IFN-α6 (Accession #P05013) were obtained from the Uniprot database. The sequence for Consensus Human Leukocyte IFN (Con-IFN) was obtained from U.S. Pat. No. 4,695,623 (incorporated by reference in its entirety). Mutations in the IFN-α2b sequence designed to attenuate the affinity for the Type I IFN receptor (IFNAR) were described in U.S. Pat. No. 9,492,562 (R149A) (incorporated by reference in its entirety) and U.S. Pat. No. 9,611,322 (A145D and A145K) (incorporated by reference in its entirety). The sequence for the anti-PD-L1 mAb (REMD290) was obtained from PCT Patent Application PCT/US2017/068369 (incorporated by reference in its entirety). The sequence for the antibody heavy chain mutations to promote heterodimer formation using “knob-into-hole” were described in, e.g., Merchant et al., Nat. Biotechnol., 16:677-681, 1998. The sequence for the antibody heavy chain mutations to promote heterodimer formation via engineered disulfide bond were described in U.S. Pat. No. 8,765,412.

Sequences for the antibody-IFN fusion proteins were constructed by adding the IFN sequence to the C-terminus of the antibody heavy chain sequence using techniques well known and understood by one of ordinary skill in the art. For example, a consensus interferon mutant molecule (SEQ ID NO: 2) was expressed as C-terminal fusion to an anti-PD-L1 antibody heavy chain (SEQ ID NO: 16) and co-expressed with an anti-PD-L1 light chain (SEQ ID NO: 15) to form an anti-PD-L1 Ab-con-IFN-α mutant fusion protein.

The gene sequence of the target proteins were optimized for mammalian expression and synthesized. The target sequences were then cloned to expression vector pTT5. 300 mL CHO-3E7 cells were then transiently transfected and cells were cultured in shaker flasks for 5-7 days. After culture cell culture medium was harvested and fusion proteins were purified with Protein A resin. Purified proteins quality was checked with SDS-PAGE and SEC-HPLC. The final material was formulated in 10 mM histidine, 8% sucrose, 0.01% polysorbate 80, pH 5.5 with concentration above 1 mg/ml and stored at −80° C.

### Example 2

### Determination of the IFN Activity of PD-L1 Ab-IFN-α Mutant Fusion Proteins Using an Interferon-α Reporter Assay

An IFN-α reporter assay system that measures expression of secreted embryonic alkaline phosphatase (SEAP) under the control of an IFN-α responsive promoter was used to determine the IFN activity of the PD-L1 Ab-IFN-α mutant fusion proteins. The HEK293-based reporter cell line was found to express low levels of PD-L1, so to assess the true non-targeted activity, REMD290 PD-L1 Ab was pre-incubated to block any PD-L1 on the cells, so the only binding was between IFN and IFNAR. To determine the targeted activity the reporter cell line was not pre-incubated with PD-L1 Ab.

The IFN-α reporter assay protocol was as follows: (1) the IFN-α reporter cells were added to 96-well plate and then incubated with or without PD-L1 Ab for 2 hrs; (2) pre-diluted fusion proteins and IFN-α2b were added to the wells and then incubated overnight; (3) QUANTI-Blue solution was added to each well and then incubated for 4 hrs; and (4) Absorbance readings were taken at 655 nm.

Twenty constructs listed in Table 5 were examined. EC50 ratio1 indicates the fold of attenuation of IFN activity compared to wild-type IFN-α2b on PD-L1-negative cells (non-targeted cells), while EC50 ratio2 represents the fold of enhancement of IFN activity compared to wild-type IFN-α2b on PD-L1-positive cells (targeted cells). Targeting index is the product of ratio1 and ratio2, suggesting the relative level of safety window of the fusion protein.

TABLE 5
Activity of PD-L1 Ab-IFN mutant fusion proteins on PD-L1-negative
and PD-L1-positive reporter cells compared to wild-type IFN-α2b

| | EC50 ratio1 | EC50 ratio2 | Targeting index |
|---|---|---|---|
| | (EC50 of FP/EC50 of IFN-α2b) | (EC50 of IFN-α2b/EC50 of FP) | (EC50 ratio1 × |
| Sample ID | on PD-L1-negative cells | on PD-L1-positive cells | EC50 ratio2) |
| FP-01 | 38.4 | 6.73 | 258 |
| FP-02 | 35658.0 | 0.08 | 2786 |
| FP-03 | 15.3 | 6.08 | 93 |
| FP-04 | 28174.0 | 0.02 | 455 |
| FP-05 | 687.0 | 1.52 | 1043 |
| FP-06 | 11.8 | 2.86 | 34 |
| FP-07 | 8441.0 | 0.17 | 1409 |
| FP-08 | 209.0 | 2.56 | 534 |
| FP-09 | 6067.0 | 0.14 | 876 |
| FP-10 | 8.7 | 3.81 | 33 |
| FP-11 | 2877.0 | 0.10 | 302 |
| FP-12 | 49059.0 | 0.01 | 386 |
| FP-13 | 85030.0 | 0.01 | 416 |
| FP-14 | 1576.0 | 0.22 | 353 |
| FP-15 | 1442.4 | 0.45 | 644 |
| FP-16 | 37.6 | 2.69 | 101 |
| FP-17 | 15.0 | 4.09 | 61 |
| FP-18 | 16093.0 | 0.04 | 630 |
| FP-19 | 33.9 | 15.69 | 531 |
| FP-20 | 1141.2 | 0.75 | 860 |
| IFN-a2b | 1.0 | 1.00 | 1 |

### Example 3

### Stability of PD-L1 Ab-IFN Mutant Fusion Proteins

The fusion proteins were stored in in 4° C. or −80° C. for 80 days and then the biological activity was measured by the reporter assay as described above. The fusion proteins retained biological activity as shown in Table 6.

TABLE 6
Activity of PD-L1 Ab-IFN mutant fusion proteins
is retained after 80 days storage at 4° C.

| | Sample | | EC50 (pM) | EC50 (pM) |
|---|---|---|---|---|
| Sample | storage | | on PD-L1- | on PD-L1- |
| ID | conditions | | negative cells | positive cells |
| FP-05 | 4° | C. | 181 | 0.342 |
| | −80° | C. | 186 | 0.367 |
| FP-07 | 4° | C. | 2963 | 3.32 |
| | −80° | C. | 2783 | 3.18 |
| FP-08 | 4° | C. | 57.6 | 0.274 |
| | −80° | C. | 81.4 | 0.292 |
| FP-09 | 4° | C. | 1598 | 4.37 |
| | −80° | C. | 2038 | 4.35 |
| IFN-α2b | −80° | C. | 0.374 | 0.437 |

### Example 4

### Expression of PD-L1 Increases the Potency of PD-L1 Ab-IFN Mutant Fusion Proteins in Tumor Cell Growth Inhibition

Tumor cell growth inhibition by IFN-α2b or the fusion proteins was measured using CellTiter 96 AQueous Non-Radioactive Cell Proliferation Assay. The protocol was as follows: (1) Daudi NIH tumor cells were added to 96-well plate and were incubated for 5 days in growth medium (cell control) or growth medium with the addition of pre-diluted IFN-α2b or fusion proteins. Growth medium without cells was used as background control; (2) MTS was added to each well and then incubated for 4 hrs; (3) The absorbance readings were taken at 490 nm; and (4) Percent maximum growth was calculated as follows: (sample signal-background signal)/(cell control signal-background signal)*100.

Parental Daudi cells do not express PD-L1. A Daudi/PD-L1 cell line expressing PD-L1 was created by transfecting the human PD-L1 gene into Daudi cells. Nine constructs listed in Table 7 were examined for growth inhibition on both Daudi cells (PD-L1-negative) and Daudi/PD-L1 cells (PD-L1-positive). EC50 ratio1, EC50 ratio2, and targeting index were defined as in Example 2. There was a large variation in targeting index among the fusion proteins tested.

TABLE 7
Activity of PD-L1 Ab-IFN mutant fusion proteins on Daudi
cells and Daudi/PD-L1 cells compared to wild-type IFN-α2b

| | EC50 ratio1 | EC50 ratio2 | |
|---|---|---|---|
| | (EC50 of FP/EC50 | (EC50 of IFN-α2b/ | |
| | of IFN-α2b) | EC50 of FP) on | Targeting Index |
| | on PD-L1-negative | PD-L1-positive | (EC50 ratio1 × |
| Sample ID | cells | cells | EC50 ratio2) |
| FP-07 | 549.1 | 0.68 | 375 |
| FP-08 | 7.7 | 0.83 | 6 |
| FP-14 | 265.4 | 1.20 | 318 |
| FP-15 | 13.4 | 1.74 | 23 |
| FP-16 | 8.5 | 0.44 | 4 |
| FP-17 | 9.5 | 0.36 | 3 |
| FP-18 | 752.2 | 1.34 | 1011 |
| FP-19 | 10.7 | 4.25 | 45 |
| FP-20 | 57.8 | 3.20 | 185 |
| IFN-α2b | 1.0 | 1.00 | 1 |

Daudi/PD-L1 #2 and Daudi/PD-L1 #4 are transfected Daudi cell lines expressing different levels of PD-L1. As depicted in Table 8, PD-L1 expression significantly enhances fusion protein inhibitory activity on Daudi cells, and the level of enhancement is correlated with PD-L1 expression level.

TABLE 8

| | PD-L1 | | | |
|---|---|---|---|---|
| | expression | EC50 (pM) | | |
| Cell line | (MFI) | IFN-α2b | FP-14 | FP-16 |
| Daudi | 4.0 | 1.06 | 357 | 9.34 |
| Daudi/PD-L1 #4 | 5.8 | 1.55 | 474 | 13.3 |
| Daudi/PD-L1 #2 | 44.9 | 1.06 | 2.53 | 2.80 |

### Example 5

### PD-L1 Ab-IFN Mutant Fusion Proteins Inhibit OVCAR3 Cell Growth

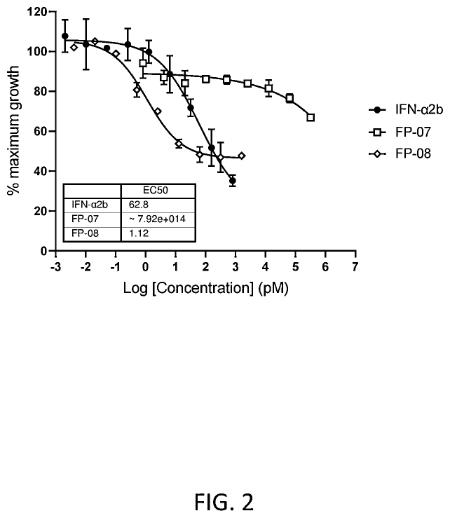

OVCAR-3 ovarian cancer cells express low levels of PD-L1. The cells were treated for 5 days at 37° C./5% CO₂ with titrated concentrations of IFN-α2b, FP-07, FP-08, or left untreated. The cell growth inhibition was measured using CellTiter 96 AQueous Non-Radioactive Cell Proliferation Assay (Promega). FP-08 treatment resulted in the strongest growth inhibition (EC50=1.12 pM vs. 62.8 pM for IFN-α2b). FP-07 treatment showed much less potent growth inhibition activity. FP-07 and FP-08 inhibit OVCAR-3 cell growth with different attenuated IFN activity (see FIG. 2).

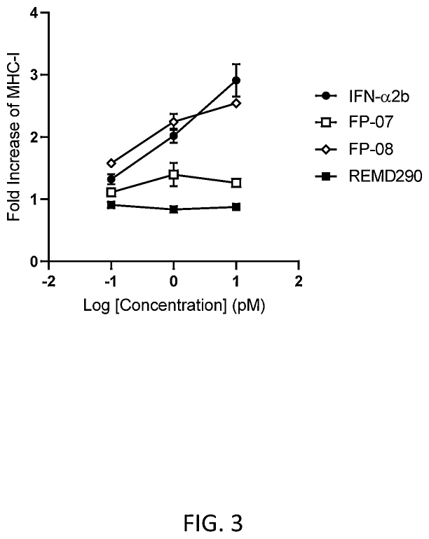

MHC-1 expression assay: OVCAR-3 cells were incubated for 2 days in growth medium (cell control) or growth medium with the addition of samples (REMD290, IFN-α2b or fusion proteins). MHC-I expression was determined by FACS with PE anti-human HLA-A,B,C antibody (sample MFI) or without antibody (background MFI). Fold increase was calculated as follows: (sample MFI-background MFI)/(cell control MFI-background MFI). Treatment with IFN-α2b and FP-08 resulted in similarly increased levels of MHC-I on the cell surface and FP-07 treatment resulted in only slightly increased MHC-I expression, compared to cell control. FP-07 and FP-08 increase MHC-I expression in OVCAR-3 cells. Increased MHC-I expression can make tumor cells more immunogenic, i.e., better recognized by CD8⁺ T cells (see FIG. 3).

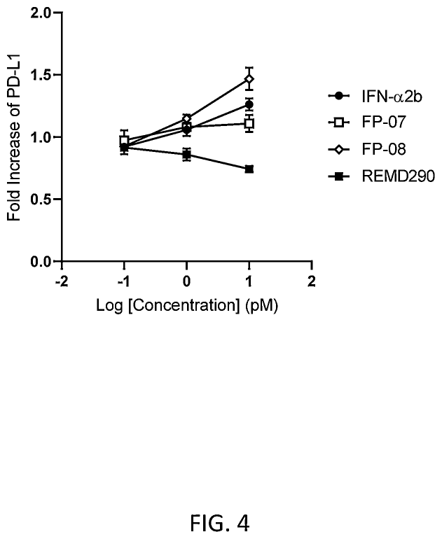

PD-L1 expression assay: OVCAR-3 cells were incubated for 2 days in growth medium (cell control) or growth medium with addition of samples (REMD290, IFN-α2b or fusion proteins). PD-L1 expression was determined by FACS with REMD290 (sample MFI) or without antibody (background MFI), and then by FITC goat anti-human IgG secondary antibody. Fold increase was calculated as follows: (sample MFI−background MFI)/(cell control MFI−background MFI). Treatment with FP-08 and IFN-α2b resulted in a minor increase in PD-L1 expression while FP-07 did not increase PD-L1 expression. Increased PD-L1 levels may help enhance the targeted effect of the PD-L1 directed fusion proteins on tumor cells (see FIG. 4).

All of the articles and methods disclosed and claimed herein can be made and executed without undue experimentation in light of the present disclosure. While the articles and methods of this disclosure have been described in terms of preferred embodiments, it will be apparent to those of skill in the art that variations may be applied to the articles and methods without departing from the spirit and scope of the disclosure. All such variations and equivalents apparent to those skilled in the art, whether now existing or later developed, are deemed to be within the spirit and scope of the disclosure as defined by the appended claims. All patents, patent applications, and publications mentioned in the specification are indicative of the levels of those of ordinary skill in the art to which the disclosure pertains. All patents, patent applications, and publications are herein incorporated by reference in their entirety for all purposes and to the same extent as if each individual publication was specifically and individually indicated to be incorporated by reference in its entirety for any and all purposes. The disclosure illustratively described herein suitably may be practiced in the absence of any element(s) not specifically disclosed herein. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

### SEQUENCE LISTINGS

The nucleic and amino acid sequences listed in the accompanying sequence listing are shown using standard letter abbreviations for nucleotide bases and one letter codes for amino acids, as defined in 37 C.F.R. 1.822.

SEQ ID NO: 1 is a wildtype consensus IFN amino acid sequence.

SEQ ID NO: 2 is a consensus IFN (R150A) mutant amino acid sequence.

SEQ ID NO: 3 is a consensus IFN (A146D) mutant amino acid sequence.

SEQ ID NO: 4 is a consensus IFN (A146K) mutant amino acid sequence.

SEQ ID NO: 5 is a consensus IFN (A146D and R150A) mutant amino acid sequence.

SEQ ID NO: 6 is a wildtype IFN-α2b amino acid sequence.

SEQ ID NO: 7 is an IFN-α2b (R149A) mutant amino acid sequence.

SEQ ID NO: 8 is a wildtype IFN-α5 amino acid sequence.

SEQ ID NO: 9 is an IFN-α5 (R150A) mutant amino acid sequence.

SEQ ID NO: 10 is an IFN-α5 (A146D) mutant amino acid sequence.

SEQ ID NO: 11 is a wildtype IFN-α6 amino acid sequence.

SEQ ID NO: 12 is an IFN-α6 (R150A) mutant amino acid sequence.

SEQ ID NO: 13 is an IFN-α6 (A146D) mutant amino acid sequence.

SEQ ID NO: 14 is an IFN-α6 (A146K) mutant amino acid sequence.

SEQ ID NO: 15 is the amino acid sequence encoding the light chain of an anti-PD-L1 antibody.

SEQ ID NO: 16 is the amino acid sequence encoding the heavy chain of an anti-PD-L1 antibody.

SEQ ID NO: 17 is the amino acid sequence encoding the heavy chain of an anti-PD-L1 antibody.

SEQ ID NO: 18 is the amino acid sequence encoding the heavy chain of an anti-PD-L1 antibody containing the “knob”.

SEQ ID NO: 19 is the amino acid sequence encoding the heavy chain of an anti-PD-L1 antibody containing the “hole”.

SEQ ID NOS: 20-23 are the amino acid sequences of peptide linkers.

SEQ ID NO: 24 is the amino acid sequence of a peptide leader sequence.

SEQUENCE LISTINGS
Wildtype Consensus IFN sequence
(SEQ ID NO: 1)
CDLPQTHSLGNRRALILLAQMRRISPFSCLKDRHDFGFPQEEFDGNQFQK
AQAISVLHEMIQQTFNLFSTKDSSAAWDESLLEKFYTELYQQLNDLEACV
IQEVGVEETPLMNVDSILAVKKYFQRITLYLTEKKYSPCAWEVVRAEIMR
SFSLSTNLQERLRRKE
Consensus IFN (R150A) mutant amino acid sequence
(SEQ ID NO: 2)
CDLPQTHSLGNRRALILLAQMRRISPFSCLKDRHDFGFPQEEFDGNQFQK
AQAISVLHEMIQQTFNLFSTKDSSAAWDESLLEKFYTELYQQLNDLEACV
IQEVGVEETPLMNVDSILAVKKYFQRITLYLTEKKYSPCAWEVVRAEIMA
SFSLSTNLQERLRRKE
Consensus IFN (A146D) mutant amino acid sequence
(SEQ ID NO: 3)
CDLPQTHSLGNRRALILLAQMRRISPFSCLKDRHDFGFPQEEFDGNQFQK
AQAISVLHEMIQQTFNLFSTKDSSAAWDESLLEKFYTELYQQLNDLEACV
IQEVGVEETPLMNVDSILAVKKYFQRITLYLTEKKYSPCAWEVVRDEIMR
SFSLSTNLQERLRRKE
Consensus IFN (A146K) mutant amino acid sequence
(SEQ ID NO: 4)
CDLPQTHSLGNRRALILLAQMRRISPFSCLKDRHDFGFPQEEFDGNQFQK
AQAISVLHEMIQQTFNLFSTKDSSAAWDESLLEKFYTELYQQLNDLEACV
IQEVGVEETPLMNVDSILAVKKYFQRITLYLTEKKYSPCAWEVVRKEIMR
SFSLSTNLQERLRRKE
Consensus IFN (A146D and R150A) mutant amino acid
sequence
(SEQ ID NO: 5)
CDLPQTHSLGNRRALILLAQMRRISPFSCLKDRHDFGFPQEEFDGNQFQK
AQAISVLHEMIQQTFNLFSTKDSSAAWDESLLEKFYTELYQQLNDLEACV
IQEVGVEETPLMNVDSILAVKKYFQRITLYLTEKKYSPCAWEVVRDEIMA
SFSLSTNLQERLRRKE
Wildtype IFN-α2b sequence
(SEQ ID NO: 6)
CDLPQTHSLGSRRTLMLLAQMRRISLFSCLKDRHDFGFPQEEFGNQFQKA
ETIPVLHEMIQQIFNLFSTKDSSAAWDETLLDKFYTELYQQLNDLEACVI
QGVGVTETPLMKEDSILAVRKYFQRITLYLKEKKYSPCAWEVVRAEIMRS
FSLSTNLQESLRSKE
IFN-α2b (R149A) mutant amino acid sequence
(SEQ ID NO: 7)
CDLPQTHSLGSRRTLMLLAQMRRISLFSCLKDRHDFGFPQEEFGNQFQKA
ETIPVLHEMIQQIFNLFSTKDSSAAWDETLLDKFYTELYQQLNDLEACVI
QGVGVTETPLMKEDSILAVRKYFQRITLYLKEKKYSPCAWEVVRAEIMAS
FSLSTNLQESLRSKE
Wildtype IFN-α5 sequence
(SEQ ID NO: 8)
CDLPQTHSLSNRRTLMIMAQMGRISPFSCLKDRHDFGFPQEEFDGNQFQK
AQAISVLHEMIQQTFNLFSTKDSSATWDETLLDKFYTELYQQLNDLEACM
MQEVGVEDTPLMNVDSILTVRKYFQRITLYLTEKKYSPCAWEVVRAEIMR
SFSLSANLQERLRRKE
IFN-α5 (R150A) mutant amino acid sequence
(SEQ ID NO: 9)
CDLPQTHSLSNRRTLMIMAQMGRISPFSCLKDRHDFGFPQEEFDGNQFQK
AQAISVLHEMIQQTFNLFSTKDSSATWDETLLDKFYTELYQQLNDLEACM
MQEVGVEDTPLMNVDSILTVRKYFQRITLYLTEKKYSPCAWEVVRAEIMA
SFSLSANLQERLRRKE
IFN-α5 (A146D) mutant amino acid sequence
(SEQ ID NO: 10)
CDLPQTHSLSNRRTLMIMAQMGRISPFSCLKDRHDFGFPQEEFDGNQFQK
AQAISVLHEMIQQTFNLFSTKDSSATWDETLLDKFYTELYQQLNDLEACM
MQEVGVEDTPLMNVDSILTVRKYFQRITLYLTEKKYSPCAWEVVRDEIMR
SFSLSANLQERLRRKE
Wildtype IFN-α6 sequence
(SEQ ID NO: 11)
CDLPQTHSLGHRRTMMLLAQMRRISLFSCLKDRHDFRFPQEEFDGNQFQK
AEAISVLHEVIQQTFNLFSTKDSSVAWDERLLDKLYTELYQQLNDLEACV
MQEVWVGGTPLMNEDSILAVRKYFQRITLYLTEKKYSPCAWEVVRAEIMR
SFSSSRNLQERLRRKE
IFN-α6 (R150A) mutant amino acid sequence
(SEQ ID NO: 12)
CDLPQTHSLGHRRTMMLLAQMRRISLFSCLKDRHDFRFPQEEFDGNQFQK
AEAISVLHEVIQQTFNLFSTKDSSVAWDERLLDKLYTELYQQLNDLEACV
MQEVWVGGTPLMNEDSILAVRKYFQRITLYLTEKKYSPCAWEVVRAEIMA
SFSSSRNLQERLRRKE
IFN-α6 (A146D) mutant amino acid sequence
(SEQ ID NO: 13)
CDLPQTHSLGHRRTMMLLAQMRRISLFSCLKDRHDFRFPQEEFDGNQFQK
AEAISVLHEVIQQTFNLFSTKDSSVAWDERLLDKLYTELYQQLNDLEACV
MQEVWVGGTPLMNEDSILAVRKYFQRITLYLTEKKYSPCAWEVVRDEIMR
SFSSSRNLQERLRRKE
IFN-α6 (A146K) mutant amino acid sequence
(SEQ ID NO: 14)
CDLPQTHSLGHRRTMMLLAQMRRISLFSCLKDRHDFRFPQEEFDGNQFQK
AEAISVLHEVIQQTFNLFSTKDSSVAWDERLLDKLYTELYQQLNDLEACV
MQEVWVGGTPLMNEDSILAVRKYFQRITLYLTEKKYSPCAWEVVRKEIMR
SFSSSRNLQERLRRKE
Amino acid sequence encoding the light chain of
an anti-PD-L1 antibody
(SEQ ID NO: 15)
DIQMTQSPSSVSASVGDRVTITCKTSQDVNTAVAWYQQKPGQAPRLLIYW
ASTRHTGVPDRFSGSGSGTDFTLTISSLQAEDVAVYYCQQHYNTPLTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
Amino acid sequence encoding the heavy chain of
an anti-PD-L1 antibody
(SEQ ID NO: 16)
QMQLVQSGAEVKKTGSSVKVSCKASGYTFTSYSINWVRQAPGKGLEWVAY
FYVGNGYTDYNEKFKGRVTMTRDTSTSTVYMELSSLRSEDTAVYYCARGG
LPYYFDYWGQGTLVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFLGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK
Amino acid sequence encoding the heavy chain of
an anti-PD-L1 antibody
(SEQ ID NO: 17)
QMQLVQSGAEVKKTGSSVKVSCKASGYTFTSYSINWVRQAPGKGLEWVAY
FYVGNGYTDYNEKFKGRVTMTRDTSTSTVYMELSSLRSEDTAVYYCARGG
LPYYFDYWGQGTLVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFLGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLG
Amino acid sequence encoding the heavy chain of
an anti-PD-L1 antibody containing the knob
(SEQ ID NO: 18)
QMQLVQSGAEVKKTGSSVKVSCKASGYTFTSYSINWVRQAPGKGLEWVAY
FYVGNGYTDYNEKFKGRVTMTRDTSTSTVYMELSSLRSEDTAVYYCARGG
LPYYFDYWGQGTLVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFLGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVYTLP
PCQEEMTKNQVSLWCLVKGFYPSDIAVEWESNGQPENNYKTTPPVLDSDG
SFFLYSRLTVDKSRWQEGNVFSCSVMHEALHNHYTQKSLSLSLG
Amino acid sequence encoding the heavy chain of
an anti-PD-L1 antibody containing the hole
(SEQ ID NO: 19)
QMQLVQSGAEVKKTGSSVKVSCKASGYTFTSYSINWVRQAPGKGLEWVAY
FYVGNGYTDYNEKFKGRVTMTRDTSTSTVYMELSSLRSEDTAVYYCARGG
LPYYFDYWGQGTLVTVSSASTKGPSVFPLAPCSRSTSESTAALGCLVKDY
FPEPVTVSWNSGALTSGVHTFPAVLQSSGLYSLSSVVTVPSSSLGTKTYT
CNVDHKPSNTKVDKRVESKYGPPCPPCPAPEFLGGPSVFLFPPKPKDTLM
ISRTPEVTCVVVDVSQEDPEVQFNWYVDGVEVHNAKTKPREEQFNSTYRV
VSVLTVLHQDWLNGKEYKCKVSNKGLPSSIEKTISKAKGQPREPQVCTLP
PSQEEMTKNQVSLSCAVKGFYPSDIAVEWESNGQPENNYKTTPPVLDSDG
SFFLVSRLTVDKSRWQEGNVFSCSVMHEALHNHYTQKSLSLSLG
Amino acid sequence of a peptide linker
(SEQ ID NO: 20)
SGGGGS
Amino acid sequence of a peptide linker
(SEQ ID NO: 21)
AEAAAKEAAAKAGS
Amino acid sequence of a peptide linker
(SEQ ID NO: 22)
GGGGGGGG
Amino acid sequence of a peptide linker
(SEQ ID NO: 23)
GGGGSGGGGSGGGGS
Amino acid sequence of a peptide leader sequence
(SEQ ID NO: 24)
MGWSWILLFLLSVTAGVHS

1-39. (canceled)

40. An isolated fusion protein comprising 1) a mutated consensus IFN-α (con-IFN-α) variant polypeptide and 2) a targeting moiety, wherein said con-IFN-α variant polypeptide demonstrates a reduced activity and affinity for the IFN-αR1 and IFN-αR2 receptor complex (IFN-αR) as compared to the polypeptide represented by SEQ ID NO: 6; wherein the targeting moiety restores the reduced affinity of the con-IFN-α variant polypeptide on targeted cells.

41. The isolated fusion protein according to claim 40, wherein the fusion molecule comprises a con-IFN-α mutant molecule having the amino acid sequence selected from the group consisting of: SEQ ID NO: 2, SEQ ID NO: 3, SEQ ID NO: 4, and SEQ ID NO: 5.

42. The isolated fusion protein according to claim 40, wherein the targeting moiety is selected from the group consisting of: a target moiety targeting a marker expressed on an IFN receptor-expressing cell; a target moiety targeting a tissue-specific marker; a target moiety targeting a cancer tissue, a target moiety targeting an HBV infected tissue, and a target moiety targeting an HCV infected tissue.

43. The isolated fusion protein according to claim 42, wherein the targeting moiety is selected from the group consisting of a fully human antibody, a humanized antibody, a chimeric antibody, a monoclonal antibody, a polyclonal antibody, a recombinant antibody, a bispecific antibody, a heterodimeric antibody, an antigen-binding antibody fragment, a Fab, a Fab′, a Fab₂, a Fab′₂, a IgG, a IgM, a IgA, a IgE, a scFv, a dsFv, a dAb, a nanobody, a unibody, and a diabody.

44. The isolated fusion protein according to claim 43, wherein the targeting moiety is selected from the group consisting of a human anti-PD-L1 Ab having the light chain sequence set forth in SEQ ID NO: 15 and the heavy chain sequence set forth in SEQ ID NO: 16, a human anti-PD-L1 Ab having the light chain sequence set forth in SEQ ID NO: 15 and the heavy chain sequence set forth in SEQ ID NO: 17, and a heterodimeric human anti-PD-L1 Ab having the light chain sequence set forth in SEQ ID NO: 15 and the heavy chain sequences set forth in SEQ ID NO: 18 and SEQ ID NO: 19.

45. The isolated fusion protein according to claim 40, wherein the fusion molecule is selected from the group consisting of an anti-PD-L1 Ab-con-IFN-α (R150A) fusion molecule comprising a light chain having the amino acid sequence set forth in SEQ ID NO: 15, and a con-IFN-α (R150A) molecule having the amino acid sequence of SEQ ID NO: 2 directly fused to the C-terminus of a heavy chain having the amino acid sequence set forth in SEQ ID NO: 16, an anti-PD-L1 Ab-con-IFN-α (A146D) fusion molecule comprising a light chain having the amino acid sequence set forth in SEQ ID NO: 15, and a con-IFN-α (A146D) molecule having the amino acid sequence of SEQ ID NO: 3 directly fused to the C-terminus of a heavy chain having the amino acid sequence set forth in SEQ ID NO: 16, an anti-PD-L1 Ab-con-IFN-α (A146K) fusion molecule comprising a light chain having the amino acid sequence set forth in SEQ ID NO: 15, and a con-IFN-α (A146K) molecule having the amino acid sequence of SEQ ID NO: 4 directly fused to the C-terminus of a heavy chain having the amino acid sequence set forth in SEQ ID NO: 16.

46. The isolated fusion protein according to claim 45, wherein the fusion molecule is an anti-PD-L1 Ab-con-IFN-α (R150A) fusion molecule comprising a light chain having the amino acid sequence set forth in SEQ ID NO: 15, and a con-IFN-α (R150A) molecule having the amino acid sequence of SEQ ID NO: 2 directly fused to the C-terminus of a heavy chain having the amino acid sequence set forth in SEQ ID NO: 16.

47. The isolated fusion protein according to claim 45, wherein the fusion molecule is an anti-PD-L1 Ab-con-IFN-α (A146D) fusion molecule comprising a light chain having the amino acid sequence set forth in SEQ ID NO: 15, and a con-IFN-α (A146D) molecule having the amino acid sequence of SEQ ID NO: 3 directly fused to the C-terminus of a heavy chain having the amino acid sequence set forth in SEQ ID NO: 16.

48. The isolated fusion protein according to claim 40, wherein the isolated fusion proteins comprise a con-IFN-α variant polypeptide that is attached to the targeting moiety via a peptide linker, wherein the peptide linker has the sequence selected from the group consisting of: SEQ ID NO: 20, SEQ ID NO: 21, SEQ ID NO: 22, and SEQ ID NO: 23.

49. The isolated fusion protein according to claim 48, wherein the fusion molecule is selected from the group consisting of an anti-PD-L1 Ab-con-IFN-α (R150A) fusion molecule comprising a light chain having the amino acid sequence set forth in SEQ ID NO: 15, and a con-IFN-α (R150A) molecule having the amino acid sequence of SEQ ID NO: 2 fused to the C-terminus of a heavy chain having the amino acid sequence set forth in SEQ ID NO: 17 using a linker having the amino acid sequence of SEQ ID NO: 22, an anti-PD-L1 Ab-con-IFN-α (R150A) fusion molecule comprising a light chain having the amino acid sequence set forth in SEQ ID NO: 15, and a con-IFN-α (R150A) molecule having the amino acid sequence of SEQ ID NO: 2 fused to the C-terminus of a heavy chain having the amino acid sequence set forth in SEQ ID NO: 17 using a linker having the amino acid sequence of SEQ ID NO: 23, an anti-PD-L1 Ab-con-IFN-α (A146D) fusion molecule comprising a light chain having the amino acid sequence set forth in SEQ ID NO: 15, and a con-IFN-α (A146D) molecule having the amino acid sequence of SEQ ID NO: 3 fused to the C-terminus of a heavy chain having the amino acid sequence set forth in SEQ ID NO: 17 using a linker having the amino acid sequence of SEQ ID NO: 22, an anti-PD-L1 Ab-con-IFN-α (A146D) fusion molecule comprising a light chain having the amino acid sequence set forth in SEQ ID NO: 15, and a con-IFN-α (A146D) molecule having the amino acid sequence of SEQ ID NO: 3 fused to the C-terminus of a heavy chain having the amino acid sequence set forth in SEQ ID NO: 17 using a linker having the amino acid sequence of SEQ ID NO: 23, an anti-PD-L1 Ab-con-IFN-α (A146D and R150A) fusion molecule comprising a light chain having the amino acid sequence set forth in SEQ ID NO: 15, and a con-IFN-α (A146D and R150A) molecule having the amino acid sequence of SEQ ID NO: 5 fused to the C-terminus of a heavy chain having the amino acid sequence set forth in SEQ ID NO: 17 using a linker having the amino acid sequence of SEQ ID NO: 22, an anti-PD-L1 Ab-con-IFN-α (R150A) heterodimeric fusion molecule comprising a light chain having the amino acid sequence set forth in SEQ ID NO: 15, and a con-IFN-α (R150A) molecule having the amino acid sequence of SEQ ID NO: 2 fused to the C-terminus of a heavy chain having the amino acid sequence set forth in SEQ ID NO: 19 using a linker having the amino acid sequence of SEQ ID NO: 22 and a second paired antibody heavy chain having the amino acid sequence of SEQ ID NO: 18 without a fused IFN, and an anti-PD-L1 Ab-con-IFN-α (A146D and R150A) heterodimeric fusion molecule comprising a light chain having the amino acid sequence set forth in SEQ ID NO: 15, and a con-IFN-α (A146D and R150A) molecule having the amino acid sequence of SEQ ID NO: 5 fused to the C-terminus of a heavy chain having the amino acid sequence set forth in SEQ ID NO: 19 using a linker having the amino acid sequence of SEQ ID NO: 22 and a second paired antibody heavy chain having the amino acid sequence of SEQ ID NO: 18 without a fused IFN.

50. A pharmaceutical composition comprising an isolated fusion protein according to claim 40 in admixture with a pharmaceutically acceptable excipient or carrier.

51. A method for treating a type I interferon-mediated disorder in a subject, comprising administering to the subject a therapeutically effective amount of the pharmaceutical composition according to claim 50; wherein the disease or disorder is selected from the group consisting of cancer, infectious diseases, immune disorders, inflammatory diseases or conditions, and autoimmune diseases.

52. The method according to claim 51, wherein the disorder is a cancer selected from the group consisting of: B cell lymphoma; a lung cancer (small cell lung cancer and non-small cell lung cancer); a bronchus cancer; a colorectal cancer; a prostate cancer; a breast cancer; a pancreas cancer; a stomach cancer; an ovarian cancer; a urinary bladder cancer; a brain or central nervous system cancer; a peripheral nervous system cancer; an esophageal cancer; a cervical cancer; a melanoma; a uterine or endometrial cancer; a cancer of the oral cavity or pharynx; a liver cancer; a kidney cancer; a biliary tract cancer; a small bowel or appendix cancer; a salivary gland cancer; a thyroid gland cancer; a adrenal gland cancer; an osteosarcoma; a chondrosarcoma; a liposarcoma; a testes cancer; and a malignant fibrous histiocytoma; a skin cancer; a head and neck cancer; lymphomas; sarcomas; multiple myeloma; and leukemias.

53. The method according to claim 52, wherein the subject has a cancer selected from the group consisting of a resistant cancer, a recurrent cancer, and a refractory cancer.

54. The method according to claim 52, wherein the method further comprises a second therapy selected from the group consisting of: cytotoxic chemotherapy, immunotherapy, small molecule kinase inhibitor targeted therapy, surgery, radiation therapy, and stem cell transplantation.

55. The method according to claim 54 wherein the immunotherapy is selected from the group consisting of: treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, PD-L1, OX-40, CD137, GITR, LAG3, TIM-3, CD40, CD47, SIRPα, ICOS, Siglec 8, Siglec 9, Siglec 15, TIGIT and VISTA; treatment using bispecific T cell engaging antibodies (BiTE®) such as blinatumomab: treatment involving administration of biological response modifiers such as TNF family, IL-1, IL-4, IL-7, IL-12, IL-15, IL-17, IL-21, IL-22, GM-CSF, IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR: TLR7, TLR8, and TLR 9) agonists CpG and imiquimod; wherein the combination therapy provides increased effector cell killing of tumor cells, i.e., a synergy exists between the isolated fusion proteins and the immunotherapy when co-administered.

56. The method according to claim 51, wherein the infectious disease is hepatitis B virus (HBV).

57. The method according to claim 56, wherein the method further comprises a second therapy selected from the group consisting of: treatment using nucleo(t)side analogs such as Tenofovir disoproxil fumarate (TDF), Tenofovir alafenamide (TAF), Lamivudine, Adefovir dipivoxil, Entecavir (ETV), Telbivudine, AGX-1009, emtricitabine, clevudine, ritonavir, dipivoxil, lobucavir, famvir, FTC, N-Acetyl-Cysteine (NAC), PC1323, theradigm-HBV, thymosin-alpha, and ganciclovir, besifovir (ANA-380/LB-80380), and tenofovir-exaliades (TLX/CMX157); treatment using other antiviral drugs such as siRNA, antisense oligonucleotides, capsid assembly modulators, and polymerase inhibitors; treatment using immunomodulatory therapies such as TLR7 agonist, TLR8 agonist, STING agonist, RIG-I activator, PD-1 blocking antibody, PD-L1 blocking antibody, TIM-3 blocking antibody, LAG-3 blocking antibody, and CTLA-4 blocking antibody; treatment using therapeutic vaccines against HBV antigens; and treatment using adoptive cellular therapy such as HBV-specific CAR-T cells, HBV-specific TCR-T cells, and other HBV-specific cellular therapies, and wherein the combination therapy provides increased antiviral activity, i.e., a synergy exists between the isolated fusion proteins and the combination therapy when co-administered.

58. The method according to claim 51, wherein the infectious disease is hepatitis C virus (HCV).

59. The method according to claim 58, wherein the method further comprises a second therapy selected from the group consisting of: protease inhibitors, polymerase inhibitors, direct-acting antivirals, ribavirin and pegylated interferon. In various embodiments, the second therapy is selected from the group consisting of: Mavyet (glecaprevir/pibrentasvir), Epclusa (sofosbuvir/velpatasvir), Vosevi (sofosbuvir/velpatasvir/voxilapresvir), Harvoni (ledipasvir/sofosbuvir), Sovaldi (sofosbuvir), and Zepatier (elbasvir/grazoprevir), and wherein the combination therapy provides increased antiviral activity, i.e., a synergy exists between the isolated fusion proteins and the combination therapy when co-administered.
